SEQUENCE LISTING (33 sequences)

SEQ ID NO: 1 - protein, 453 aa, Artificial Sequence
  Synthetic Sequence
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, Artificial Sequence
  Synthetic Sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 124 aa, Artificial Sequence
  Synthetic Sequence
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 4 - protein, 107 aa, Artificial Sequence
  Synthetic Sequence
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 5 - protein, 5 aa, Artificial Sequence
  Synthetic Sequence
YYGIS

SEQ ID NO: 6 - protein, 17 aa, Artificial Sequence
  Synthetic Sequence
WISTYNGNTHYARMLQG

SEQ ID NO: 7 - protein, 15 aa, Artificial Sequence
  Synthetic Sequence
ENYYGSGAYRGGMDV

SEQ ID NO: 8 - protein, 11 aa, Artificial Sequence
  Synthetic Sequence
RASQSVSSYLA

SEQ ID NO: 9 - protein, 7 aa, Artificial Sequence
  Synthetic Sequence
DASNRAT

SEQ ID NO: 10 - protein, 9 aa, Artificial Sequence
  Synthetic Sequence
QQRSNWPWT

SEQ ID NO: 11 - protein, 124 aa, Artificial Sequence
  Synthetic Sequence
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGSYRGGMDVWGQGTTVTVSS

SEQ ID NO: 12 - protein, 124 aa, Artificial Sequence
  Synthetic Sequence
QVQLVQSGAEVKKPGASVKVSCKASGYDFTYYGISWVRQAPGQGLEWMGWISTYNGNKHYARMLQGRVTMTTDTSTRTAYMELRSLRSDDTAVYYCARENYYGSGSYRGGMDVWGQGTTVTVSS

SEQ ID NO: 13 - protein, 124 aa, Artificial Sequence
  Synthetic Sequence
QVQLVQSGAEVKKPGASVKVSCKASGYDFTYYGISWVRQAPGQGLEWMGWISTYNGGTHYARMLQGRVTMTTDTSTRTAYMELRSLRSDDTAVYYCARENYYGSGSYRGGMDVWGQGTTVTVSS

SEQ ID NO: 14 - protein, 124 aa, Artificial Sequence
  Synthetic Sequence
QVQLVQSGAEVKKPGASVKVSCKASGYDFTYYGISWVRQAPGQGLEWMGWISTYNGVTHYARMLQGRVTMTTDTSTRTAYMELRSLRSDDTAVYYCARENYYGSGSYRGGMDVWGQGTTVTVSS

SEQ ID NO: 15 - protein, 124 aa, Artificial Sequence
  Synthetic Sequence
QVQLVQSGAEVKKPGASVKVSCKASGYDFTYYGISWVRQAPGQGLEWMGWISTYNGGTHYARMLQGRVTMTTDTSTRTAYMELRSLRSDDTAVYYCARENYYGSGAYRGGMDAWGQGTTVTVSS

SEQ ID NO: 16 - protein, 124 aa, Artificial Sequence
  Synthetic Sequence
QVQLVQSGAEVKKPGASVKVSCKASGYDFTYYGISWVRQAPGQGLEWMGWISTYNGVTHYARMLQGRVTMTTDTSTRTAYMELRSLRSDDTAVYYCARENYYGSGAYRGGMDAWGQGTTVTVSS

SEQ ID NO: 17 - protein, 124 aa, Artificial Sequence
  Synthetic Sequence
QVQLVQSGAEVKKPGASVKVSCKASGYDFTYYGISWVRQAPGQGLEWMGWISTYNGKTHYARMHQGRVTMTTDTSTRTAYMELRSLRSDDTAVYYCARENYYGSGAYRGGMDAWGQGTTVTVSS

SEQ ID NO: 18 - protein, 124 aa, Artificial Sequence
  Synthetic Sequence
QVQLVQSGAEVKKPGASLKVSCKASGYPFTNYGISWVRQAPGQGLEWMGWISTYNGNTHYAQKLQGRVTMTTDTSTTTAYMDLRSLRSDDTAVYYCARENYYGSGSYRGGMDVWGQGTTVTVSS

SEQ ID NO: 19 - protein, 124 aa, Artificial Sequence
  Synthetic Sequence
QVQLVQSGAEVKKPGASVKVSCKASGYSFTTYGISWVRQAPGQGLEWMGWISTYNGNTHYAQKLQGRVTMTTDTSTRTAYMELRSLRSDDTAVYYCARENYYGSGSYRGGMDVWGQGTTVTVSS

SEQ ID NO: 20 - protein, 121 aa, Artificial Sequence
  Synthetic Sequence
QVQLVQSGAEVKKPGASVKVSCKASGYTFTSYGINWVRQAPGQGLEWMGWISTYNGNTNSAQKLQGRVTMTTDTSTSTAYMELRSLRSDDTAVYYCARAHSSSWFDAFDIWGQGTMVTVSS

SEQ ID NO: 21 - protein, 126 aa, Artificial Sequence
  Synthetic Sequence
QVQLVESGGGVVQPGRSLRLSCAASGFTFSSFAMHWVRQAPGKGLEWVALIPFDGSSNYYADSVKGRFTISRDNSKNTLYLQMNSLRAEDTAVYYCARDRNYYGSGSFSFDAFDIWGQGTLVTVSS

SEQ ID NO: 22 - protein, 126 aa, Artificial Sequence
  Synthetic Sequence
QVQLVESGGGVVQPGRSLRLSCAASGFTFSNYALHWVRQAPGKGLEWVALISYDGSDKYYADSVKGRFAISRDNSKNTLYLQMNSLRAEDTAVYYCARDREYCTYSSCSYDAFDIWGQGTMVTVSS

SEQ ID NO: 23 - protein, 121 aa, Artificial Sequence
  Synthetic Sequence
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGYSSAWFDAFDIWGQGTMVTVSS

SEQ ID NO: 24 - protein, 107 aa, Artificial Sequence [sequence omitted]

SEQ ID NO: 25 - protein, 107 aa, Artificial Sequence
  Synthetic Sequence
DIQMTQSPSSLSASVGDRVTITCRASQGISNWLAWYQQKPEKAPKSLIYAASSLQSGVPSRFSGSGSGTDFTLTISSLQPEDFATYYCQQYNSYPYTFGQGTKLEIK

SEQ ID NO: 26 - protein, 107 aa, Artificial Sequence
  Synthetic Sequence
DIQMTQSPSSLSASVGDRVTITCRASQGISSWLAWYQQKPEKAPKSLIYAASSLQSGVPSRFSGSGSGTDFTLTISSLQPEDYATYYCQQYNSYPYTFGQGTKLEIK

SEQ ID NO: 27 - protein, 107 aa, Artificial Sequence [sequence omitted]

SEQ ID NO: 28 - protein, 121 aa, Artificial Sequence
  Synthetic Sequence
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 29 - protein, 106 aa, Artificial Sequence
  Synthetic Sequence
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 30 - protein, 449 aa, Artificial Sequence
  Synthetic Sequence
QVQLVQSGAEVKKPGASVKVSCKASGYTFTSYDINWVRQAPGQGLEWMGWLNPNSGNTGYAQKFQGRVTMTRNTSISTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 216 aa, Artificial Sequence
  Synthetic Sequence
QSVLTQPPSVSGAPGQRVTISCTSSSSDIGAGLGVHWYQQLPGTAPKLLIYGYYNRPSGVPDRFSGSKSGTSASLTITGLLPEDEGDYYCQSYDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 119 aa, Artificial Sequence
  Synthetic Sequence
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 33 - protein, 111 aa, Artificial Sequence
  Synthetic Sequence
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

(19) United States
(12) Patent Application Publication
(10) Pub. No.: US 2023/0235070 A1
(43) Pub. Date: Jul. 27, 2023

(54) METHOD OF TREATMENT OF INFLAMMATORY BOWEL DISEASE USING ANTI-TL1A ANTIBODIES

(71) Applicant: Pfizer Inc., New York, NY (US)

(72) Inventors: Mary Lynn BANIECKI, Boston, MA (US); Mina HASSAN-ZAHRAEE, Cambridge, MA (US); Kenneth Eugene HUNG, Watertown, MA (US); Gang LI, Phoenixville, PA (US); Li XI, Chestnut Hill, MA (US); Zhan YE, Lexington, MA (US)

(21) Appl. No.: 18/002,368

(22) PCT Filed: Jun. 23, 2021
(86) PCT No.: PCT/IB2021/055546

§ 371 Date: Dec. 19, 2022

Related U.S. Application Data
(60) Provisional application No. 63/044,390, filed on Jun. 26, 2020.

(51) Int. Cl. C07K 16/28; A61P 1/12

(52) U.S. Cl. CPC C07K 16/2875; A61P 1/12

(57) ABSTRACT

The present invention relates to a method for treating inflammatory bowel disease (IBD) in a patient, the method comprising administering to the patient an anti-TNF-like ligand 1A (TL1A) antibody in an induction dosing regimen sufficient to improve signs and symptoms of IBD by at least 12 weeks after the start of treatment with the anti-TL1A antibody, said induction dosing regimen comprising a plurality of individual induction doses, wherein the method further comprises administering to the patient a subsequent maintenance dosing regimen after completion of the induction dosing regimen, said maintenance dosing regimen comprising a plurality of individual maintenance doses separated from each other by at least 2 weeks.

### FIELD

The present invention relates to the treatment of signs and symptoms of inflammatory bowel disease with an anti-tumor necrosis factor-like ligand 1A (TL1A) antibody.

### BACKGROUND

Inflammatory bowel disease (IBD), which encompasses Crohn's disease and ulcerative colitis (UC), is a chronic inflammatory disorder affecting ˜1.6 million people in the USA, and ˜2.5-3 million people in Europe. To induce remission in patients with moderately to severely active UC, treatment recommendations include appropriate doses of oral corticosteroids, biologic therapies such as the tumor necrosis factor inhibitors (TNFi) infliximab (monotherapy or in combination with azathioprine), adalimumab, ustekinumab, golimumab, the integrin receptor antagonist vedolizumab, and the oral, small molecule Janus kinase inhibitor tofacitinib (Rubin et al, 2019 Am J Gastroenterol; 114:384-413). However, non-response and loss of response to treatments have been observed in UC and, therefore, the development of novel treatments for UC and IBD is still an unmet clinical need (Lichtenstein et al 2018 Am J Gastroenterol; 113:481-517).

A range of mucosal immune system components—including epithelial cells, innate and adaptive immune cells, cytokines and chemokines—contribute to the pathogenesis of IBD (Wallace et al, 2014, World J Gastroenterol; 20:6-21). One of the immune components involved in the pathogenesis of IBD is TNF-like ligand 1A (TL1A, or tissue necrosis factor superfamily member 15 (TNFSF15)). Genome-wide association studies have linked TNFSF15 single nucleotide polymorphisms with disease severity; for example, an association was observed between the r511554257 single nucleotide polymorphism and medically refractory UC compared with healthy controls (Haritunians et al, 2010, Inflammatory bowel diseases; 16:1830-1840). TL1A has been found to be upregulated in IBD tissue specimens, with level of expression corresponding to the severity of disease (Bamias et al, 2010, Clin Immunol; 137:242-249).

T cell-mediated signaling and cytokine production, for example, T helper (Th)1 cells producing interferon-γ, Th17 cells producing interleukin (IL)-6 and IL-17, and Th2 cells producing IL-4 and IL-13 are costimulated by TL1A binding to death receptor 3, (Migone et al, 2002, Immunity 16; 16:479-492; Takedatsu et al, 2008, Gastroenterology 135; 552-567; Meylan et al 2011; Mucosal Immunity 4; 172-185; Meylan et al 2008; Immunity 29; 79-89). Because increased cytokine production leads to chronic inflammation, inhibition of TL1A may be a therapeutic target for inflammatory diseases, including IBD.

UC is chronic inflammatory disease of the large intestine characterized by diffuse mucosal inflammation. The underlying pathophysiology of this disease results from the interplay of genetic susceptibility in immune genes and alterations in the gut microbiome. Current treatments for UC target immune cell activation including non-selective medicines such as corticosteroids, mesalamine, thiopurines as well as selective biologic (anti-TNFa, anti-a4b7, and anti-IL-12/23 agents) and small molecule Janus kinase inhibitors. Despite these treatments, many patients have an inadequate response as determined by endoscopy and develop refractory disease. There is, therefore, an urgent need to develop therapeutics driving gastrointestinal healing determined by, e.g., endoscopy (referred to as “endoscopic healing”) and to define tissue, blood, and microbiome biomarkers to help guide therapy.

There is still an unmet need for an effective, safe, and well tolerated treatment in subjects with moderate to severe ulcerative colitis. The hallmark clinical symptoms of UC include bloody diarrhoea associated with rectal urgency and tenesmus. The clinical course is marked by exacerbation and remission. The diagnosis of UC is suspected on clinical grounds and supported by diagnostic testing, and elimination of infectious causes (Dignass et al., J Crohn's Colitis. 2012; 6(10):965-90). The most severe intestinal manifestations of UC are toxic megacolon and perforation. Extraintestinal complications include arthritis (peripheral or axial involvement), dermatological conditions (erythema nodosum, aphthous stomatitis, and pyoderma gangrenosum), inflammation of the eye (uveitis), and liver dysfunction (primary sclerosing cholangitis). Subjects with UC are at an increased risk for colon cancer, and the risk increases with the duration of disease as well as extent of colon affected by the disease (Rutter et al, 2004 Gastroenterology; 126(2):451-9).

The aim of medical treatment in UC is to control inflammation and reduce symptoms. Available pharmaceutical therapies are limited, do not always completely abate the inflammatory process and may have significant adverse effects. Therapies for mild to moderate active UC include 5-aminosalicylic acid derivatives and immunosuppressants.

Genome-wide association studies have linked over 200 genes with IBD, with many of these genes associated with this underlying dysregulated immunoregulatory functions (de Lange et al 2017, Nat Genet.; 49(2): 256-610). One of the strongest genetic variants associated with IBD exist in the Tumor Necrosis Factor Superfamily member 15 (TNFSF15) locus (Siakavellas et al 2015; Inflamm. Bowel Dis. 21 (10): 2441-52). Variants in TNFSF15 have been linked to the pathogenesis of several autoimmune diseases—including psoriasis, rheumatoid arthritis, and multiple sclerosis—implicating a broad role for TNFSF15 in human inflammatory diseases. In IBD, TNFSF15 variants may confer higher risk for more aggressive, penetrating, fibrostenotic, and perianal disease complications (Yang et al 2014 J Crohns Colitis; 8(10): 1315-26; Tung et al 2014 J Gasteroenterol Hepatol; 29(4):273-9). TNFSF15 encodes for the protein TNF-like ligand 1A (TL1A), which is highly expressed in human colonic tissue during active colitis (Bamias et al 2013; Curr Opinion Gasteroenterol. 29(6):597-602).

The mechanistic impact of TL1A in pre-clinical models is pleiotropic. While early studies have shown a pathogenic role for TL1A overexpression in driving inflammatory Th1, Th17, and group 2 innate lymphoid cell responses, more recent reports in mouse models of acute colitis and ileitis revealed a contrasting protective role for endogenous TL1A in supporting anti-inflammatory T regulatory cells (Treg) and group 3 innate lymphoid cell function (Prehn et al, 2004, Clin Immunol 112(1):66-77; Castellanos, et al 2019; Mucosal Immunol. 11(5): 1466-1476). In addition to the impact on lymphoid cells, mouse models have revealed a key impact of TL1A overexpression in intestinal fibrosis. Moreover, in vitro studies of peripheral blood macrophage revealed a contribution of the TNFSF15 risk haplotype in synergistically regulating NOD2 ligand induced inflammatory cytokines (Hedl and Abraham, 2014; PNAS 111 (37) 13451-13456. Collectively, these pre-clinical studies highlight a potential central homeostatic role for TL1A in modulating selective innate and adaptive immune pathways critical for IBD, as well as, the key clinical complication of fibrosis. To date, no studies have defined the mechanisms underlying the potential efficacy of anti-TL1A therapy in humans.

Pre-clinical studies in rodent colitis models and human cells have shown that anti-TL1A antibodies can reduce tissue fibrosis, the number of fibroblasts, and clinical disease score, thereby highlighting their potential as biologic therapies for IBD (Clarke et al 2002, MAbs 10:664-677; Shih et al 2014 Mucosal Immunol; 7:1492-1503).

PF-06480605 is a first-in-class, fully human immunoglobulin G1 monoclonal antibody that targets TL1A. A first-in-human study of PF-06480605 in healthy participants demonstrated a favorable safety profile and target engagement, at single intravenous (IV) doses of up to 800 mg and IV doses of PF-06480605 500 mg, or subcutaneous (SC) doses up to 300 mg administered once every 2 weeks (Q2W) for a total of 3 doses (Banfield et al 2019, Br J Clin Pharmacol Epub ahead of print). The aim of this Phase 2a, multicenter, single-arm study was to evaluate the safety, tolerability, and efficacy of PF-06480605 in its first use in participants with moderate-to-severe UC. Here, we provide analysis of tissue transcriptional, peripheral blood proteomics and fecal metagenomic data of participants treated with anti-TL1A PF-06480605. The results identify the selective reduction in tissue Th17 and fibrosis pathways as targets of anti-TL1A therapy in patients achieving endoscopic improvement. Correlative changes in the blood proteome reflect tissue and systemic changes in participants achieving endoscopic improvement. Anti-TL1A therapy alters the intestinal microbiome, as characterized by a reduction in pathobionts associated with the UC microbiome. Furthermore, our results identify both genetic variants and tissue gene signatures that predict response. These results provide the first mechanistic insights underlying the inhibition of TL1A in human IBD. Further, the results may also inform a precision medicine approach for clinical management based on anti-TL1A therapy.

### SUMMARY OF THE INVENTION

The invention provides methods of treating a patient with inflammatory bowel disease (IBD) comprising administering an anti-TNF-like ligand 1A (TL1A) antibody. Those skilled in the art will recognize, or be able to ascertain using no more than routine experimentation, many equivalents to the specific embodiments of the invention described herein. Such equivalents are intended to be encompassed by the following embodiments (E).

E1. In a first embodiment, the invention relates to a method for treating inflammatory bowel disease (IBD) in a patient, the method comprising administering to the patient an anti-TNF-like ligand 1A (TL1A) antibody in an induction dosing regimen sufficient to improve signs and symptoms of IBD by at least 12 weeks after the start of treatment with the anti-TL1A antibody, said induction dosing regimen comprising a plurality of individual induction doses, wherein the method further comprises administering to the patient a subsequent maintenance dosing regimen after completion of the induction dosing regimen, said maintenance dosing regimen comprising a plurality of individual maintenance doses separated from each other by at least 2 weeks.
E2. A method as set forth in E1, wherein one or more of the individual maintenance doses are administered at least 4 weeks apart.
E3. A method as set forth in E1-E2, wherein one or more of the individual maintenance doses are administered at least 8 weeks apart.
E4. A method as set forth in E1-E3, wherein one or more of the individual maintenance doses are administered at least 12 weeks apart.
E5. A method for treating inflammatory bowel disease (IBD) in a patient, the method comprising administering to the patient an anti-TNF-like ligand 1A (TL1A) antibody in a therapeutic dosing regimen sufficient to improve signs and symptoms of IBD by at least 12 weeks after the start of treatment with the anti-TL1A antibody, said induction dosing regimen comprising a plurality of individual induction doses, wherein the method further comprises administering to the patient a subsequent maintenance dosing regimen after completion of the induction dosing regimen, said maintenance dosing regimen comprising a plurality of individual maintenance doses separated from each other by at least 1 month.
E6. A method as set forth in E5, wherein one or more of the individual maintenance doses are administered at least 2 months apart.
E7. A method as set forth in E5-E6, wherein one or more of the individual maintenance doses are administered at least 3 months apart.
E8. A method as set forth in E5-E7, wherein one or more of the individual maintenance doses are administered at least 4 months apart.
E9. A method as set forth in E5-E8, wherein one or more of the individual maintenance doses are administered at least 6 months apart.
E10. A method as set forth in E1-E9, wherein one or more of the individual maintenance doses are about 100% of the individual induction dose.
E11. A method as set forth in E1-E10, wherein one or more of the individual maintenance doses are no more than about 75% of the individual induction doses.
E12. A method as set forth in E1-E11, wherein one or more of the individual maintenance doses are no more than about 50% of the individual induction doses.
E13. A method as set forth in E1-E12, wherein one or more of the individual maintenance doses are no more than about 40% of the individual induction doses.
E14. A method as set forth in E1-E13, wherein one or more of the individual maintenance doses are no more than about 25% of the individual induction doses.
E15. A method as set forth in E1-E14, wherein one or more of the individual maintenance doses are no more than about 20% of the individual induction doses.
E16. A method as set forth in any one of E1-E15, wherein one or more of the individual induction dose is about 500 mg via intravenous injection.
E17. A method as set forth in any one of E1-E16, wherein one or more of the individual induction doses are separated from each other by 2 weeks.
E18. A method for treating inflammatory bowel disease (IBD) in a patient sufficient to improve signs and symptoms of IBD, the method comprising administering to the patient an anti-TNF-like ligand 1A (TL1A) antibody in an induction dosing regimen, said induction dosing regimen comprising a plurality of individual induction doses of 500 mg every 2 weeks via intravenous injection.
E19. The method according to E1-E18, wherein the induction dosing regimen is continued for at least 12 weeks.
E20. The method as set forth in any one of E1-19, wherein the maintenance dosing regimen is maintained for at least 2 months.
E21. The method as set forth in any one of E1-20, wherein the maintenance dosing regimen is maintained for at least 3 months.
E22. The method as set forth in any one of E1-21, wherein the maintenance dosing regimen is maintained for at least 4 months.
E23. The method as set forth in any one of E1-22, wherein the maintenance dosing regimen is maintained for at least 6 months.
E24. The method as set forth in any one of E1-E23, wherein following the induction dosing regimen the patient experiences an improvement in signs and symptoms of IBD characterized by a clinical response.
E25. The method as set forth in any one of E1-E24, wherein following the induction dosing regimen the patient experiences an improvement in signs and symptoms of IBD characterized by an endoscopic response.
E26. The method as set forth in any one of E1-E25, wherein following the induction dosing regimen the patient experiences an improvement in signs and symptoms of IBD characterized by a clinical remission.
E27. The method as set forth in any one of E1-E26, wherein following the induction dosing regimen the patient experiences an improvement in signs and symptoms of IBD characterized by an endoscopic remission.
E28. The method as set forth in any one of E1-E27, wherein following the induction dosing regimen the patient experiences an improvement in signs and symptoms of IBD characterized by a deep remission.
E29. The method as set forth in any one of E1-E28, wherein following the induction dosing regimen the patient experiences an improvement in signs and symptoms of IBD characterized by a symptomatic remission.
E30. The method as set forth in any one of E1-E29, wherein following the induction dosing regimen the patient experiences an improvement in signs and symptoms of IBD characterized by an endoscopic improvement.
E31. The method as set forth in any one of E1-E30, wherein following the induction dosing regimen the patient experiences an improvement in signs and symptoms of IBD that are maintained while the patient receives the maintenance dosing regimen.
E32. The method according to E1-E23, wherein the induction dosing regimen with the anti-TL1A antibody effectively improves signs and symptoms of IBD by at least 14 weeks after starting of treatment with the anti-TL1A antibody.
E33. The method according to E1-E32, wherein the improvement in signs and symptoms of IBD is characterized by an improvement in the Mayo endoscopic subscore.
E34. The method according to E1-E33, wherein the improvement in signs and symptoms of IBD is characterized by a reduction of the patient's Mayo endoscopic subscore by at least one integer.
E35. The method according to E1-E34, wherein the improvement in signs and symptoms of IBD is characterized by a reduction of the patient's Mayo endoscopic subscore by at least two integers.
E36. The method according to E1-E35, wherein the improvement in signs and symptoms of IBD is characterized by a reduction of the patient's Mayo endoscopic subscore by at least three integers.
E37. The method according to E1-E36, wherein the improvement in signs and symptoms of IBD is characterized by the patient having a Mayo endoscopic subscore of 0 or 1.
E38. The method according to E1-E37, wherein the improvement in signs and symptoms of IBD is characterized by the patient having a total Mayo score of 0, 1, 2, or 3.
E39. The method according to E1-E38, wherein the improvement in signs and symptoms of IBD is characterized by the patient having a total Mayo score of 0, 1, or 2.
E40. The method according to E1-E39, wherein the improvement in signs and symptoms of IBD is characterized by the patient having a total Mayo score of 0 or 1
E41. The method according to E1-E40, wherein the improvement in signs and symptoms of IBD is characterized by the patient having a RHI Robarts Histopathology Index of less than 5.
E42. The method according to E1-E41, wherein the improvement in signs and symptoms of IBD is characterized by the patient having a Geboes Index of less than 3.2.
E43. The method according to E1-E42, wherein the improvement in signs and symptoms of IBD is maintained for during the maintenance dosing regimen for at least 2 months.
E44. The method according to E1-E43, wherein the improvement in signs and symptoms of IBD is maintained during the maintenance dosing regimen for at least 3 months.
E45. The method according to E1-E44, wherein the improvement in signs and symptoms of IBD is maintained during the maintenance dosing regimen for at least 4 months.
E46. The method according to E1-E45, wherein the improvement in signs and symptoms of IBD is maintained during the maintenance dosing regimen for at least 6 months.
E47. The method according to E1-E46, wherein the improvement in signs and symptoms of IBD is maintained during the maintenance dosing regimen for at least 12 months.
E48. A method for treating inflammatory bowel disease (IBD) in a patient, the method comprising administering to the patient an anti-TN F-like ligand 1A (TL1A) antibody in an induction dosing regimen sufficient to improve signs and symptoms of IBD by at least 12 weeks after the start of treatment with the anti-TL1A antibody, said induction dosing regimen comprising 6 individual induction doses each of 500 mg administered 2 weeks apart, wherein the method further comprises administering to the patient a subsequent maintenance dosing regimen after completion of the induction dosing regimen, said maintenance dosing regimen comprising a plurality of individual maintenance doses, each individual maintenance dose being no more than 75% of the individual induction dose, and wherein each individual maintenance dose is separated from each other by at least 4 weeks.
E49. The method according to E1-E48, wherein the patient was previously treated with corticosteroids prior to administering the anti-TL1A antibody.
E50. The method according to E1-E49, wherein the patient was previously treated with one or more treatments selected from the group consisting of tumor necrosis factor inhibitors, anti-integrins, azathioprine, 6-mercaptopurine, and methotrexate.
E51. The method according to E1-E50, wherein the patient shows a reduction of fecal calprotectin from baseline of at least 50% from week 2 to week 26 of treatment.
E52. The method according to E1-E51, wherein the patient shows a reduction of fecal calprotectin from baseline of at least 60% from week 2 to week 26 treatment.
E53. The method according to E1-E52, wherein the patient shows a reduction of hsCRP from baseline from week 2 to week 26 of treatment.
E54. The method according to E1-E53, wherein the IBD is ulcerative colitis (UC).
E55. The method according to E1-E54, wherein the anti-TL1A antibody comprises three CDRs from the variable heavy chain region having the sequence shown in SEQ ID NO: 1 and three CDRs from the variable light chain region having the sequence shown in SEQ ID NO: 2.
E56. The method according to E1-E55, wherein the anti-TL1A antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.
E57. The method according to E1-E56, wherein the anti-TL1A antibody comprises a variable heavy chain region having the sequence shown in SEQ ID NO: 1 and a variable light chain region having the sequence shown in SEQ ID NO: 2.
E58. The method according to E1-E57, wherein the anti-TL1A antibody comprises a heavy chain having the sequence shown in SEQ ID NO: 9 and a light chain having the sequence shown in SEQ ID NO: 10, wherein the C-terminal lysine (K) of the heavy chain amino acid sequence of SEQ ID NO: 9 is optional.
E59. The method according to E1-E58, wherein the TL1A antibody comprises a VH encoded by the nucleic acid sequence of the insert of the vector deposited as 1D1 1.31 VH having ATCC accession number PTA-120639 and a VL encoded by the nucleic acid sequence of the insert of the vector deposited as 1D11.31 VL having ATCC accession number PTA-120640.
E60. The method according to E1-E59, wherein the anti-TL1A antibody competes for binding with an anti-TL1A antibody comprising a variable heavy chain region having the sequence shown in SEQ ID NO: 1 and a variable light chain region having the sequence shown in SEQ ID NO: 2.
E61. The method according to E1-E60, wherein the anti-TL1A antibody competes for binding with an antibody comprising a VH encoded by the nucleic acid sequence of the insert of the vector deposited as 1D11.31 VH having ATCC accession number PTA-120639 and a VL encoded by the nucleic acid sequence of the insert of the vector deposited as 1D11.31 VL having ATCC accession number PTA-120640.
E62. The method according to E1-E61, wherein the anti-TL1A antibody comprises sequence pairs selected from the group consisting of SEQ ID NO:4 and 11; SEQ ID NO:4 and 12; SEQ ID NO:4 and 13; SEQ ID NO:4 and 14; SEQ ID NO:4 and 15; SEQ ID NO:4 and 16; SEQ ID NO:4 and 17; SEQ ID NO:4 and 18; SEQ ID NO:4 and 19; SEQ ID NO:20 and 24; SEQ ID NO:21 and 25; SEQ ID NO:22 and 26; SEQ ID NO:23 and 27; SEQ ID NO:28 and 29; SEQ ID NO:30 and 31; and SEQ ID NO:30 and 31.
E63. The method according to E1-E62, further comprising the steps of:
a) determining the expression level of one or more candidate genes in a sample from the patient,
b) identifying that the sample contains an abnormal expression level of the one of more candidate gene,
c) administering the induction dosing regimen or individual induction dose of the anti-TL1A antibody to a patient.
   E64. A method for treating a patient with an anti-TNF-like ligand 1A (TL1A) antibody, wherein the patient is suffering from inflammatory bowel disease (IBD), the method comprising the steps of:
a) determining whether the patient has an abnormal expression level of a one or more candidate gene by obtaining or having obtained a sample from the patient;
b) performing or having performed an assay on the sample to determine if the patient expresses abnormal levels of the one or more candidate gene;
wherein if the sample contains abnormal levels of the one or more candidate gene then administering an induction dosing regimen or induction dose of the anti-TL1A antibody to the patient, and wherein the risk of the patient being non-responsive to an induction dosing regimen or individual induction dose of anti-TL1A antibody is lower in a patient with abnormal levels of the one or more candidate gene.
   E65. A method for treating inflammatory bowel disease (IBD) in a patient, the method comprising the steps of:
a) determining the expression level of one or more candidate genes in a sample from the patient,
b) identifying that the sample contains an abnormal expression level of the one of more candidate gene,
c) administering an induction dosing regimen or individual induction dose of an anti-TNF-like ligand 1A (TL1A) antibody to the patient.
   E66. The method as set forth in E63-65, wherein the one or more candidate genes is selected from the group consisting of SOWAHB, COLCA2, TBX20, FRZB, HOXB5, NET1, FOXD2, DESI1, PARK2, PKDREJ, IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L.
   E67. The method as set forth in E63-66, wherein the one or more candidate genes are selected from the group consisting of SOWAHB, COLCA2, TBX20, FRZB, HOXB5, NET1, FOXD2, DESI1, PARK2, and PKDREJ.
   E68. The method as set forth in E63-67, wherein the one or more candidate genes comprises SOWAHB.
   E69. The method as set forth in E63-68, wherein the one or more candidate genes comprises SOWAHB, and at least one or more candidate genes selected from the group consisting of COLCA2, TBX20, FRZB, HOXB5, NET1, FOXD2, DESI1, PARK2, and PKDREJ.
   E70. The method as set forth in E63-69, wherein the one or more candidate genes comprises SOWHAB and COLCA2, and at least one or more candidate genes selected from the group consisting of SOWAHB, COLCA2, TBX20, FRZB, HOXB5, NET1, FOXD2, DESI1, PARK2, and PKDREJ.
   E71. The method as set forth in E63-70, wherein the one or more candidate genes comprises SOWAHB, COLCA2, and TBX20 and at least one or more candidate genes selected from the group consisting of FRZB, HOXB5, NET1, FOXD2, DESI1, PARK2, and PKDREJ.
   E72. The method as set forth in E63-71, comprising two or more candidate genes selected from the group consisting of SOWAHB, COLCA2, TBX20, FRZB, HOXB5, NET1, FOXD2, DESI1, PARK2, and PKDREJ.
   E73. The method as set forth in E63-72, comprising three or more candidate genes selected from the group consisting of SOWAHB, COLCA2, TBX20, FRZB, HOXB5, NET1, FOXD2, DESI1, PARK2, and PKDREJ.
   E74. The method as set forth in E63-73, comprising four or more candidate genes selected from the group consisting of SOWAHB, COLCA2, TBX20, FRZB, HOXB5, NET1, FOXD2, DESI1, PARK2, and PKDREJ.
   E75. The method as set forth in E63-74, comprising five or more candidate genes selected from the group consisting of SOWAHB, COLCA2, TBX20, FRZB, HOXB5, NET1, FOXD2, DESI1, PARK2, and PKDREJ.
   E76. The method as set forth in E63-75, comprising six or more candidate genes selected from the group consisting of SOWAHB, COLCA2, TBX20, FRZB, HOXB5, NET1, FOXD2, DESI1, PARK2, and PKDREJ.
   E77. The method as set forth in E63-76, comprising seven or more candidate genes selected from the group consisting of SOWAHB, COLCA2, TBX20, FRZB, HOXB5, NET1, FOXD2, DESI1, PARK2, and PKDREJ.
   E78. The method as set forth in E63-77, comprising eight or more candidate genes selected from the group consisting of SOWAHB, COLCA2, TBX20, FRZB, HOXB5, NET1, FOXD2, DESI1, PARK2, and PKDREJ.
   E79. The method as set forth in E63-78, comprising nine or more candidate genes selected from the group consisting of SOWAHB, COLCA2, TBX20, FRZB, HOXB5, NET1, FOXD2, DESI1, PARK2, and PKDREJ.
   E80. The method as set forth in E63-79, comprising the candidate genes of SOWAHB, COLCA2, TBX20, FRZB, HOXB5, NET1, FOXD2, DESI1, PARK2, and PKDREJ.
   E81. The method as set forth in E63-E80, wherein the abnormal expression level of the one or more candidate gene is based on the one or more candidate gene's level of mRNA or expressed protein.
   E82. The method as set forth in E63-E80, wherein the abnormal expression level of the one or more candidate gene is based on the one or more candidate gene's mRNA levels.
   E83 The method as set forth in E63-E82, wherein the expression level of the one or more candidate gene is compared against a baseline expression level which is based on the expression level of the one or more candidate gene for a healthy individual who is not suffering from IBD or UC.
   E84. The method as set forth in E63-E82, wherein the expression level of the one or more candidate gene is compared against a baseline expression level which is based on an estimated expression level for individuals who are non-responsive to anti-TL1A antibody treatment.
   E85. The method as set forth in E63-E84, wherein the abnormal expression level of the one or more candidate gene is at least 50% greater or lesser from the baseline level.
   E86. The method as set forth in E63-E85, wherein the abnormal expression level of the one or more candidate gene is at least 2-fold greater or lesser from the baseline level.
   E87. The method as set forth in E63-E86, wherein the abnormal expression level of the one or more candidate gene is at least 10-fold greater or lesser from the baseline level.
   E88. The method as set forth in E63-E87, wherein the abnormal expression level of the one or more candidate gene is at least 100-fold greater or lesser from the baseline level.
   E89. The method as set forth in E63-E88, wherein the abnormal expression level of the one or more candidate gene is at least 1000-fold greater or lesser from the baseline level.
   E90. The method as set forth in E63-89, wherein the one or more candidate genes are selected from the group consisting of IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L.
   E91. The method as set forth in E63-90, wherein the one or more candidate genes are selected from the group consisting of IL-1B, IL-23A, IFNG, IL-12RB1, IL-21R, IRF4, and BATF.
   E92. The method as set forth in E63-91, wherein the one or more candidate genes are selected from the group consisting of CD80/86, HLA-DRB5/DQB1/DRB1, HLA-DRA, CD40, and ICOS.
   E93. The method as set forth in E63-92, wherein the one or more candidate genes are selected from the group consisting of MMP3, MMP7, MMP10 and CHI3L.
   E94. The method as set forth in E63-93, wherein the abnormal expression level is an elevated level, and the one or more of the candidate genes is selected from the group consisting of SOWAHB, COLCA2, FRZB, HOXB5, NET1, FOXD2, PARK2, and PKDREJ.
   E95. The method as set forth in E63-94, wherein the abnormal expression level is a decreased level, and the one or more of the candidate genes is selected from the group consisting of TBX20 and DESI1.
   E96. The method as set forth in E63-95, wherein the sample is a tissue sample.
   E97. The method as set forth in E63-96, wherein the sample is a tissue sample from a site of IBD inflammation.
   E98. The method as set forth in E63-95, wherein the sample is a peripheral blood sample.
   E99. The method as set forth in E63-95, wherein the sample is an intestinal biopsy sample.
   E100. The method as set forth in E1-E99, wherein the patient is haplotype A or haplotype C.
   E101. A method for treating a patient with an anti-TNF-like ligand 1A (TL1A) antibody as set forth in E1-E100, wherein the patient is suffering from inflammatory bowel disease (IBD), the method comprising the steps of:
a) determining whether the patient is a haplotype A, B or C for TNFSF15 by obtaining or having obtained a biological sample from the patient;
b) performing or having performed a genotyping assay on the biological sample to determine if the patient is of haplotype A, B or C for TNFSF15;
c) wherein the risk of the patient being non-responsive to the induction dosing regimen or individual induction dose of anti-TL1A antibody is lower in a patient of haplotype A or haplotype C than in a patient of haplotype B;
d) wherein if the patient is of haplotype B for TNFSF15 then administering a maintenance dosage regimen of the anti-TL1A antibody to the patient that provides an increased individual maintenance dose relative to the individual maintenance dose provided to patients of haplotype A or C.
   E102. A method for treating a patient with an anti-TNF-like ligand 1A (TL1A) antibody as set forth in E1-E101, wherein the patient is suffering from inflammatory bowel disease (IBD), the method comprising the steps of:
a) determining whether the patient is a haplotype A, B or C for TNFSF15 by obtaining or having obtained a biological sample from the patient;
b) performing or having performed a genotyping assay on the biological sample to determine if the patient is of haplotype A, B or C for TNFSF15;
c) wherein the risk of the patient being non-responsive to the induction dose of anti-TL1A antibody is lower in a patient of haplotype A or haplotype C than in a patient of haplotype B;
d) wherein if the patient is of haplotype B for TNFSF15 then administering a maintenance dosage regimen of the anti-TL1A antibody to the patient that provides a decreased time interval between the individual maintenance doses relative to the time intervals between individual maintenance doses provided to patients of haplotype A or C.
   E103. The method as set forth in E1-E102, further comprising the steps of:
a) determining the level of one or more candidate bacterial strains in a stool sample from the patient,
b) identifying that the stool sample contains an elevated level of the one of more candidate bacterial strains,
c) administering the induction dose of the anti-TL1A antibody to a patient.
   E104. A method for treating inflammatory bowel disease (IBD) in a patient, the method comprising the steps of:
a) determining the level of one or more candidate bacterial strains in a stool sample from the patient,
b) identifying that the stool sample contains an elevated level of the one of more candidate bacterial strains,
c) administering an induction dose of an anti-TNF-like ligand 1A (TL1A) antibody to a patient.
   E105. The method as set forth in E103-104, wherein the candidate bacterial strain is selected from the group consisting of Streptococcus salivarius, Streptococcus. parasanguinis, and Haemophilus parainfluenzae.
   E106. The method as set forth in E1-E105, further comprising the steps of:
d) determining the level of one or more candidate bacterial strains in a stool sample from the patient,
e) identifying that the stool sample contains a decreased level of the one of more candidate bacterial strains,
f) administering the induction dose of the anti-TL1A antibody to a patient.
   E107. A method for treating inflammatory bowel disease (IBD) in a patient, the method comprising the steps of:
a) determining the level of one or more candidate bacterial strains in a stool sample from the patient,
b) identifying that the stool sample contains a decreased level of the one of more candidate bacterial strains,
c) administering an induction dose of an anti-TNF-like ligand 1A (TL1A) antibody to a patient.
   E108. The method as set forth in E106-107, wherein the candidate bacterial strain is selected from the group consisting of Ruminococcus albus, Ruminococcus callidus, Ruminococcus bromii, Ruminococcus gnavus, and Bifidobacterium bifidum.
   E109. The method as set forth in E103-E108, wherein the level of the one or more candidate bacterial strains is compared against a baseline bacterial level which is based on the level of the one or more candidate bacterial strain for a healthy individual who is not suffering from IBD or UC.
   E110. The method as set forth in E103-E108, wherein the level of the one or more candidate bacterial strains is compared against a baseline bacterial level which is based on an estimated level of those candidate bacterial strains for individuals who are non-responsive to anti-TL1A antibody treatment.
   E111. The method as set forth in E109-110, wherein the level of the one or more candidate bacterial strains is at least 50% greater or lesser from the baseline bacterial level.
   E112. The method as set forth in E109-110, wherein the level of the one or more candidate bacterial strains is at least 2-fold greater or lesser from the baseline bacterial level.
   E113. The method as set forth in E109-110, wherein the level of the one or more candidate bacterial strains is at least 10-fold greater or lesser from the baseline bacterial level.
   E114. The method as set forth in E109-110, wherein the level of the one or more candidate bacterial strains is at least 100-fold greater or lesser from the baseline bacterial level.
   E115. The method as set forth in E109-110, wherein the level of the one or more candidate bacterial strains is at least 1000-fold greater or lesser from the baseline bacterial level.
   E116. The method as set forth in E1-E115, further comprising treatment with an IL-23 antagonist.
   E117. A method for identifying a patient having inflammatory bowel disease as being likely to benefit from initial or continued treatment with anti-TL1A antibody treatment, and optionally treating said patient, wherein said method comprises:
(a) identifying a patient as containing an abnormal level of one or more candidate genes, selected from the group consisting of SOWAHB, COLCA2, TBX20, FRZB, HOXB5, NET1, FOXD2, DESI1, PARK2, PKDREJ, IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L;
(b) administering or having administered to said patient an anti-TL1A antibody under conditions wherein one or more selected from the group consisting of inflammatory macrophages, TH17, ILC3, OX40, OX40L, IFNγ, ILC2, IL-13, MMP, tissue remodeling, fibrosis, the intestinal population of S. salivarius, the intestinal population of S. parasanguinis, and the intestinal population of H. parainfluenzae in said patient is reduced after said administering.
   E118. The method as set forth in any one of E1-E117, wherein the patient has moderate to severe ulcerative colitis.
   E119. Use of a compound for the preparation of a medicament for the treatment of IBD according to any of E1-E118.

### BRIEF DESCRIPTION OF THE FIGURES/DRAWINGS

FIG. 1. Study design

Based on the results of an interim analysis conducted on the 12 participants at the end of the first stage (per Simon's two-stage design), the study did not meet the futility criteria, and therefore enrollment continued into the second stage.

IV, intravenous; Q2W, every 2 weeks.

FIG. 2. Participant disposition

AE, adverse event; IV, intravenous; Q2W, every 2 weeks.

FIG. 3. Mean partial Mayo score over time in participants treated with PF-06480605 500 mg IV Q2W (FAS with observed cases)

Triangles represent the mean, and circles represent outliers. Median, 25%, and 75% quartiles are shown, with whiskers indicating the last points±1.5×the interquartile range. Outliers were defined as any points below the 25% quartile−1.5×the interquartile range or above the 75% quartile+1.5×the interquartile range.

FAS, full analysis set; IV, intravenous; Q2W, every 2 weeks.

FIG. 4: Anti-TL1a demonstrates target engagement in serum and tissue (4A): Serum total TL1A levels were measured at baseline in endoscopic improvement responders (R) (18) and non-responders (NR) (32) and at week 14 in endoscopic improvement R (18) and NR (29). Serum total TL1A increased post therapy in both endoscopic improvement R (P<0.001) and NR (P<0.001). Serum total TL1A are not significantly different between R and NR at both Pre-Therapy and Post-Therapy. (4B) Tissue TL1A levels were measured at baseline in endoscopic improvement R (18) and NR (32) and at week 14 in endoscopic improvement R (16) and NR (30). Tissue TL1A decreased post therapy in both endoscopic improvement R (P<0.001) and NR (P<0.001). Tissue TL1A was not significantly different between R and NR at both pre-therapy and post-therapy, respectively. The R and NR with averages and standard errors were intentionally displayed side by side for both serum and tissue TL1A measured at pre- and post-therapy to provide easy and clear comparison.

FIG. 5: Precision medicine potential mechanism of action for PF-06480605. Without wishing to be bound by any particular theory, Proposed mechanisms of action for PF-06480605 are as follows: (1) Inflammatory macrophages (MO) are increased in IBD and produce IL23, IL1B, TL1A. IL1B can feed back in an autocrine fashion to promote cytokine production; 2) TL1A stimulates pathogenic Th17 and the data disclosed herein suggest it also mediates ILC3 and regulates Th1 through either OX40/OX40L or via the transitioning of ILC3 to ILC1 and production of interferon gamma (IFNg); 3) TL1A and 1L33 regulate ILC2 which contributes to the Th2-driven 1L13 response, MMP activation, tissue remodeling and fibrosis; 4) TL1A stimulates fibroblast proliferation and contributes to fibrosis; and/or 5) blocking TL1A inhibited inflammation, MO requirement and fibrosis.

### DETAILED DESCRIPTION OF THE INVENTION

This Phase 2a, multicenter, single-arm study of PF-06480605 500 mg IV Q2W in participants with moderate-to-severe UC was designed to analyze primary, secondary, and exploratory endpoints while allowing for maximal efficacy. The duration of the induction period (12 weeks) was chosen to increase the likelihood of participants achieving EI and was supported by non-clinical toxicology. Furthermore, assessment at Week 14 following the end of treatment with PF-06480605 is in line with other biological therapies.

In this study, PF-06480605 was generally well-tolerated and demonstrated an acceptable safety profile. Approximately two thirds of participants (66.0%) experienced TEAEs, and 6.0% experienced SAEs. Other than UC, the most common TEAEs were arthralgia (12.0%) and abdominal pain, nausea, nasopharyngitis, pharyngitis, back pain, and alopecia areata (all 6.0%). The safety and tolerability of PF-06480605 in this study were also similar to those observed in healthy participants receiving SC doses of up to 300 mg or an IV dose of 500 mg Q2W for a total of 3 doses.

Statistically significant EI (38.2% of participants) and remission (24.0% of participants) at Week 14 was observed in participants with moderate-to-severe active UC, and endoscopic remission and clinical response were achieved by 10.0% and 72.0% of participants, respectively. The placebo-adjusted treatment effect of PF-06480605 500 mg IV Q2W was supported by propensity score weighting analyses comparing Week 8 data in participants receiving placebo from two Phase 3 induction studies of tofacitinib 10 mg. Although direct comparisons cannot be made due to differences in sample size and study design, similar remission rates have been observed in participants treated with TNFi and the integrin receptor antagonist vedolizumab; for example, 27.5-33.9% of participants receiving infliximab were in remission at Week 8, 16.5% of participants receiving adalimumab were in remission at Week 8, 35.8-39.7% of participants receiving golimumab were in remission at Week 30, and 29.3% of participants receiving vedolizumab were in remission at Week 14. The durable decrease in mean change from baseline in partial Mayo score from the cessation of treatment at Week 12 and through the Week 26 follow-up suggests that decreased TL1A inhibition may be required for chronic maintenance of remission in future studies.

Histologic disease activity in UC is thought to be a predictor of clinical outcomes. In the recent VARSITY study, which compared vedolizumab and adalimumab, minimal histologic disease activity at Week 52, as indicated by RHI <5, was observed in 42.3% of participants in the vedolizumab group, and in 25.6% in the adalimumab group, whereas minimal histologic disease activity at Week 52, as indicated by a GI <3.2, was observed in 33.4% of participants in the vedolizumab group, and in 13.7% in the adalimumab group. By comparison, in the current TUSCANY study, minimal histologic activity was demonstrated in 33.3% and 47.6% in participants by RHI ≤5 and GI ≤3.2, respectively, after only 12 weeks of treatment. Therefore, it is highly plausible that the rates of minimal histologic activity would be even greater after 52 weeks of treatment with PF-06480605.

The 3-month follow-up period allowed for the measurement of PK and immunogenicity parameters. The 500 mg IV Q2W dose was chosen with the assumption that PK is similar in healthy participants and those with moderate-to-severe UC. In a previous study in healthy participants, PF-06480605 PK was observed to be typical of immunoglobulin G1 monoclonal antibodies, and modeling predicted target engagement would be maintained for the duration of dosing based on a site of action model. The model predicted that PF-06480605 500 mg Q2W would maintain sTL1A neutralization, with a 90% sTL1A coverage (P₉₀) for 87.2% of participants, assuming that 100% of participants developed ADA.

Target engagement was observed through treatment-dependent differences in sTL1A concentration. Another anti-TL1A antibody, developed by Clarke and colleagues, has demonstrated target engagement in vitro. It has been hypothesized that in the current study the PF-06480605-mediated TL1A signaling inhibition could ameliorate IBD symptoms. Decreases from baseline in fecal calprotectin and hsCRP support these efficacy results. Despite the observed trend of lower sTL1A target engagement in ADA- and NAb-positive participants, there were no statistically significant effects of ADA and NAb status on efficacy (data not shown). However, due to small sample size and high variability in observed PK and sTL1A between participants, immunogenicity would need to be studied further in a larger participant sample in longer duration studies to determine the impact on PK, target engagement and clinical response.

A one-arm study design was chosen to maximize the efficiency of recruitment. EI by central reading was chosen as the primary efficacy endpoint, to mitigate the lack of a placebo arm, as this is an objective endpoint with a well-characterized placebo response rate. However, the increased attractiveness of this study design may have allowed for a selection bias towards participants with more severe UC. Whereas the true magnitude of the efficacy response would have been best evaluated in a placebo-controlled trial, the concordance with histologic response supports further investigation of anti-TL1A treatment in subsequent clinical trials. The short duration of the study and the single IV dose meant that the long-term effects of PF-06480605 treatment on safety and efficacy in participants with moderate-to-severe UC, as well as dose-response, target coverage, immunogenicity, and SC administration were not explored. Longer-term studies in a larger participant sample are required to confirm the safety, tolerability, and efficacy profile observed in this study.

PF-06480605 demonstrated an acceptable safety and tolerability profile, with statistically significant EI and minimal histologic activity after 12 weeks of treatment. These findings warrant further study of PF-06480605 and TL1A inhibition in patients with IBD, and perhaps other inflammatory diseases involving TL1A-mediated pathogenesis.

### Biomarkers

TL1A has emerged as a central target for IBD therapy with pleiotropic effects in regulating both adaptive and innate immunity in pre-clinical models. However, the underlying mechanistic basis for anti-TL1A treatment in human IBD has not been elucidated. The present invention provide the first human data highlighting the impact of anti-TL1A therapy in regulating the Th17 and Th1 tissue cytokine response. Concordant with early in vitro and animal model data (Prehn, et al, 2004, Clin Immunol; 112(1): 66-77; Comminelli et al, 2013, Curr Opin Gastroenterol 29, 597-602), the present invention reveals a robust and selective impact of anti-TL1A on Th17 regulated genes in the human colonic tissue following treatment.

The present invention reveals that anti-TL1A therapy also regulates innate myeloid cell immunity in humans. In particular, the present invention shows that IL-1B is a significant transcriptional target of anti-TL1A therapy. These findings are concordant with mechanistic in vitro data in which autocrine TL1A signaling in macrophages regulates non-canonical IL-1B. Moreover, CytoReason analysis highlights tissue macrophages and DCs as central target of anti-TL1A therapy. Recent studies have highlighted the impact of TL1A signaling in innate lymphoid cells, particularly ILC2 and ILC3s (Meylan et al 2014, Mucosal Immunol; 7(4): 958-968; Castellanos and Longman 2019, J Clin Invest; 129:2640-2650).

Type 2 cytokines (including IL-5 and IL-13) have been identified as targets of constitutive TL1A overexpression and may reflect ILC2 tissue source in either the small intestine or the lung (Meylan et al 2014, Mucosal Immunol; 7(4): 958-968). Similarly, reduction in peripheral blood IL-9 may reflect TL1A impact on allergic Th9 disease (Cite Richard et al 2015, J Immunol; 194:3567-82). TL1A-driven activation of ILC2-produced IL-13 drives intestinal inflammation in animal models. In addition, TL1A regulates ILC3 effector function including IL-22, GMCSF, and OX40L regulation of Th1. Although our tissue analysis did not identify a specific impact on ILCs, the robust reduction of IL-5 and IL-13 in the peripheral blood may reflect tissue ILC2 effects of anti-TL1A therapy. This will be relevant for IBD as well as other tissue allergic and inflammatory diseases. Additional high-resolution studies are needed to define the impact of anti-TL1A therapy in situ.

Fibrotic complications remain a major clinical challenge in IBD, and our data support the potential role for anti-TL1A therapy in reducing tissue fibrosis. TL1A expression is associated with fibrostenosing Crohn's disease and can activate fibroblasts directly to stimulate a fibrosis associated with inflammation (Meylan 2011; Mucosal Immunol; 4(2): 172-185, Shih et al 2011, PLoS One; 6(1): e16090). Anti-TL1A blockade in pre-clinical studies reversed established fibrosis (Shih et al 2014, Mucosal Immunol 2014; 7:1492-503) and blocked progression of fibrosis in transfer T cell colitis model (Li, et al 2018, Pathol Res Pract; 214:217-227). Although our clinical trial was too short to determine impact on intestinal fibrosis, our results provide the first in human data highlighting the reduction of genes associated with remodeling of extracellular matrix and fibrosis following anti-TL1A treatment. In addition, significant reduction in IL-13 in the peripheral blood may reflect the ability of anti-TL1A therapy to retard downstream of IL-13 in pre-clinical models (Meylan et al 2011, Mucosal Immunol 2011; 4(2): 172-185). Future studies should include tissue IHC metrics to assess collagen deposition in both UC and Crohn's.

IBD is associated with distinct changes in the intestinal microbiome. Studies show that both adherent-invasive E. coli and Haemophilus parainfluenzae are increased in patients with active IBD disease and may contribute mechanistically to the inflammatory response (Gevers et al 2014, Cell Host Microbe 2014; 15:382-392). In addition, oral microbes including Haemophilus (Said et al 2013, Int J Inflam; 2013:581409) and Streptococcus subspecies show increased colonization during IBD and may contribute to an inflammatory immune response (Atarashi at al 2017, Science; 358:359-365). In contrast, strict anaerobic metabolism is reflected in the pathways of bacteria, including Ruminococcus and Bifidobacterium, associated with health, such as the production of short chain fatty acids (REF). Higher relative abundance of anaerobes Faecalibacterium and Ruminococcaceae are seen in subjects in remission after week 6 of Ustekinumab therapy compared to those with active disease (Doherty et al 2018, mBio; 9:e02120-17). Our results highlight a reduction in the opportunist pathobionts associated with IBD including S. salivarius, S. parasanguinis, and H. parainfluenza following anti-TL1A treatment. This microbial predictive signature may reflect a selective effective of TL1A in promoting anaerobicity of the intestine that may be mechanistically distinct (yet synergistic) with IL-23 blockade or unique to UC treatment. Further studies are needed to assess the taxonomic and functional metabolic consequences of these responses to aid in diagnosis and treatment.

A major goal of emerging therapy is to define biomarkers in participants at baseline that may help to stratify and maximize therapeutic efficacy while minimizing risk. Our study identifies two potential biomarkers for precision medicine strategies to optimize clinical management using anti-TL1A treatment. First, TNFSF15 haplotype analysis showed an increased likelihood of response in patients with the risk genotype. Despite the strong correlation of TNFSF15 variants with IBD, the functional impact of these SNP haplotype is not well defined. Although we could not detect the impact of this haplotype on peripheral TL1A or tissue TNFSF15 expression, the correlation with increased tissue cytokine expression may reflect disease with increased TL1A/IL1B/NOD2 synergy. Second, predictive modeling allowed us to define a 10-gene signature capable of stratifying response in this cohort. This gene pathways highlight a potential underlying role for epithelial cell function in guiding the efficacy of anti-TL1A therapy for UC that will be evaluated in future studies. These data reveal the potential for participant genetics and baseline transcriptomics to guide response to therapy.

Furthermore, our results highlight the potential role for peripheral blood biomarkers in monitoring endoscopic improvement. In particular, peripheral IL-17A not only strongly correlates with endoscopic improvement, but also reflects the biology underlying the tissue transcriptional reduction in Th17 related genes. These findings may inform the clinical use of combination anti-TL1A-based therapeutic regimens to maximize efficacy. Indeed, previous reports have highlighted the synergy of TL1A with IL-23A in regulating effector cytokines (Longman JEM 2014). The observed co-incident reduction of IL-23A following anti-TL1A therapy suggests the potential for therapeutic combination and/or bi-specific therapy.

Collectively, these findings provide the first in-human data defining the mechanism of anti-TL1A therapy in the treatment of UC and highlight the potential for companion diagnostics based on transcriptional signatures, blood based biomarkers, host genetics and the microbiome in guiding the use of anti-TL1A therapy in the treatment of IBD. Precision medicine putative mechanism of action for PF 06480605 was hypothesized from the results of this study (FIG. 7).

### General Techniques

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following terms, unless otherwise indicated, shall be understood to have the following meanings:

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a target (e.g., PD-1) epitope is an antibody that binds this epitope with greater affinity, avidity, more readily, and/or with greater duration than it binds to other target epitopes or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining beneficial or desired clinical results. For purposes of this invention, beneficial or desired clinical results include reduction or improvement in signs and symptoms of osteoarthritis, for example as compared to before administration of the anti-TL1A antibody.

“Ameliorating” means a lessening or improvement of one and more signs or symptoms of osteoarthritis, for example as compared to not administering an anti-TL1A antibody as described herein. “Ameliorating” also includes shortening or reduction in duration of a symptom.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates signs or symptoms of IBD, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more signs or symptoms of IBD, decreasing the dose of other medications required to treat the disease, enhancing the effect of another medication, and/or delaying the progression of the disease in patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. Treatment “effectively improves” or “effectively reduces” when assessment of the sign or symptom of IBD is quantified via a clinical measure relative to baseline and during and/or after the treatment period. The difference between the clinical measure at baseline and during/after treatment is compared and used to determine whether the sign or symptom has improved and the treatment is effective. This comparison can include comparison to placebo or to one or more of the prior therapies.

The term “mucosal healing” refers to a Mayo endoscopy subscore 0 or 1 and Geboes histology score 0 or 1. Aranzazu, J-E., et al. Journal of Crohn's and Colitis, Volume 11(3), 2017, 305-313.

A “patient”, an “individual” or a “subject”, used interchangeably herein, is a mammal, more preferably, a human. Mammals also include, but are not limited to, farm animals (e.g., cows, pigs, horses, chickens, etc.), sport animals, pets, primates, horses, dogs, cats, mice and rats.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

The term “subcutaneous administration” refers to the administration of a substance into the subcutaneous layer.

The term “preventing” or “prevent” refers to (a) keeping a disorder from occurring or (b) delaying the onset of a disorder or onset of symptoms of a disorder.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present invention. The materials, methods, and examples are illustrative only and not intended to be limiting.

### Anti-TL1A Antibodies

TABLE 1
Sequences of exemplary antibodies of the invention.

| SEQ ID NO: | SEQUENCE |
|---|---|
| 1 | QVQLVQSGAE VKKPGASVKV SCKAS [symbol] WVRQA PGQGLEWMGW |
| HC of PF-06480605 | ISTYNGNTHY ARMLQGRVTM TTDTSTRTAY MELRSLRSDD TAVYYCAREN |
| | YYGSGAYRGG MDVWGQGTTV TVSSastkgp svfplapssk stsggtaalg |
| | clvkdyfpep vtvswnsgal tsgvhtfpav lqssglysls svvtvpsssl |
| | gtqtyicnvn hkpsntkvdk kvepkscdkt htcppcpape aagapsvflf |
| | ppkpkdtlmi srtpevtcvv vdvshedpev kfnwyvdgve vhnaktkpre |
| | eqynstyrvv svltvlhqdw lngkeykckv snkalpapie ktiskakgqp |
| | repqvytlpp sreemtknqv sltclvkgfy psdiavewes ngqpennykt |
| | tppvldsdgs fflyskltvd ksrwqqgnvf scsvmhealh nhytqkslsl |
| | spg |
| 2 | EIVLTQSPAT LSLSPGERAT LSCRASQSVS SYLAWYQQKP GQAPRLLIYD |
| LC of PF-06480605 | ASNRATGIPA RFSGSGSGTD FTLTISSLEP EDFAVYYCQQ RSNWPWTFGQ |
| | GTKVEIKrtv aapsvfifpp sdeqlksgta svvcllnnfy preakvqwkv |
| | dnalqsgnsq esvteqdskd styslsstlt lskadyekhk vyacevthqg |
| | lsspvtksfn rgec |
| 3 | QVQLVQSGAE VKKPGASVKV SCKAS [symbol] WVRQA PGQGLEWMGW |
| VH of PF-06480605 | ISTYNGNTHY ARMLQGRVTM TTDTSTRTAY MELRSLRSDD TAVYYCAREN |
| | YYGSGAYRGG MDVWGQGTTV TVSS |
| 4 | EIVLTQSPAT LSLSPGERAT LSCRASQSVS SYLAWYQQKP GQAPRLLIYD |
| VL of PF-06480605 | ASNRATGIPA RFSGSGSGTD FTLTISSLEP EDFAVYYCQQ RSNWPWTFGQ |
| | GTKVEIK |
| 5 | [symbol] |
| CDR H1 of PF- | |
| 06480605 | |
| 6 | WISTYNGNTH YARMLQG |
| CDR H2 of PF- | |
| 06480605 | |
| 7 | ENYYGSGAYR GGMDV |
| CDR H3 of PF- | |
| 06480605 | |
| 8 | RASQSVSSYL A |
| CDR L1 of PF- | |
| 06480605 | |
| 9 | DASNRAT |
| CDR L2 of PF- | |
| 06480605 | |
| 10 | QQRSNWPWT |
| CDR L3 of PF- | |
| 06480605 | |
| 11 | QVQLVQSGAEVKKPGASVKVSCKASGYDFTYYGISWVRQAPGQGLEWMGWISTYNG |
| 1D1-1.27_VH | NTHYARMLQGRVTMTTDTSTRTAYMELRSLRSDDTAVYYCARENYYGSGSYRGGMD |
| | VWGQGTTVTVSS |
| 12 | QVQLVQSGAEVKKPGASVKVSCKASGYDFTYYGISWVRQAPGQGLEWMGWI |
| 1D1-1.28_VH | STYNGNKHYARMLQGRVTMTTDTSTRTAYMELRSLRSDDTAVYYCARENYY |
| | GSGSYRGGMDVWGQGTTVTVSS |
| 13 | QVQLVQSGAEVKKPGASVKVSCKASGYDFTYYGISWVRQAPGQGLEWMGWI |
| 1D1-1.29_VH | STYNGGTHYARMLQGRVTMTTDTSTRTAYMELRSLRSDDTAVYYCARENYY |
| | GSGSYRGGMDVWGQGTTVTVSS |
| 14 | QVQLVQSGAEVKKPGASVKVSCKASGYDFTYYGISWVRQAPGQGLEWMGWI |
| 1D1-1.30_VH | STYNGVTHYARMLQGRVTMTTDTSTRTAYMELRSLRSDDTAVYYCARENYY |
| | GSGSYRGGMDVWGQGTTVTVSS |
| 15 | QVQLVQSGAEVKKPGASVKVSCKASGYDFTYYGISWVRQAPGQGLEWMGWI |
| 1D1-1.32_VH | STYNGGTHYARMLQGRVTMTTDTSTRTAYMELRSLRSDDTAVYYCARENYY |
| | GSGAYRGGMDAWGQGTTVTVSS |
| 16 | QVQLVQSGAEVKKPGASVKVSCKASGYDFTYYGISWVRQAPGQGLEWMGWI |
| 1D1-1.33_VH | STYNGVTHYARMLQGRVTMTTDTSTRTAYMELRSLRSDDTAVYYCARENYY |
| | GSGAYRGGMDAWGQGTTVTVSS |
| 17 | QVQLVQSGAEVKKPGASVKVSCKASGYDFTYYGISWVRQAPGQGLEWMGWI |
| 1D1-1.34_VH | STYNGKTHYARMHQGRVTMTTDTSTRTAYMELRSLRSDDTAVYYCARENYY |
| | GSGAYRGGMDAWGQGTTVTVSS |
| 18 | QVQLVQSGAEVKKPGASLKVSCKASGYPFTNYGISWVRQAPGQGLEWMGWI |
| 15A9_VH | STYNGNTHYAQKLQGRVTMTTDTSTTTAYMDLRSLRSDDTAVYYCARENYY |
| | GSGSYRGGMDVWGQGTTVTVSS |
| 19 | QVQLVQSGAEVKKPGASVKVSCKASGYSFTTYGISWVRQAPGQGLEWMGWI |
| 15C11_VH | STYNGNTHYAQKLQGRVTMTTDTSTRTAYMELRSLRSDDTAVYYCARENYY |
| | GSGSYRGGMDVWGQGTTVTVSS |
| 20 | QVQLVQSGAEVKKPGASVKVSCKASGYTFTSYGINWVRQAPGQGLEWMGWI |
| 7D4_VH | STYNGNTNSAQKLQGRVTMTTDTSTSTAYMELRSLRSDDTAVYYCARAHSS |
| | SWFDAFDIWGQGTMVTVSS |
| 21 | QVQLVESGGGWQPGRSLRLSCAASGFTFSSFAMHWVRQAPGKGLEWVALI |
| 26B11_VH | PFDGSSNYYADSVKGRFTISRDNSKNTLYLQMNSLRAEDTAVYYCARDRNY |
| | YGSGSFSFDAFDIWGQGTLVTVSS |
| 22 | QVQLVESGGGWQPGRSLRLSCAASGFTFSNYALHWVRQAPGKGLEWVALI |
| 9B3_VH | SYDGSDKYYADSVKGRFAISRDNSKNTLYLQMNSLRAEDTAVYYCARDREY |
| | CTYSSCSYDAFDIWGQGTMVTVSS |
| 23 | QVQLVQSGAEVKKPGASVKVSCKASGYTFTSYAMHWVRQAPGQRLEWMGW1 |
| 22F9_VH | NAGNGNTKYSQKFQGRVTITRDTSASTAYMELSSLRSEDTAVYYCARGYSS |
| | AWFDAFDIWGQGTMVTVSS |
| 24 | AIQLTQSPSSLSASVGDRVTITCRASQGISSALAWYQQKPGKAPKLLIYDA |
| 7D4_VL | SSLESGVPSRFSGSGSGTDFTLTISSLQPEDFATYYCQQFNSYPLTFGGGT |
| | KVEIK |
| 25 | DIQMTQSPSSLSASVGDRVTITCRASQGISNWLAWYQQKPEKAPKSLIYTVX. |
| 26B11_VL | SSLQSGVPSRFSGSGSGTDFTLTISSLQPEDFATYYCQQYNSYPYTFGQGT |
| | KLEIK |
| 26 | DIQMTQSPSSLSASVGDRVTITCRASQGISSWLAWYQQKPEKAPKSLIYAA |
| 9B3_VL | SSLQSGVPSRFSGSGSGTDFTLTISSLQPEDYATYYCQQYNSYPYTFGQGT |
| | KLEIK |
| 27 | AIQLTQSPSSLSASVGDRVTITCRASQGISSALAWYQQKPGKAPKLLIYDA |
| 22F9_VL | SSLESGVPSRFSGSGSGTDFTLTISSLQPEDFATYYCQQFNSYPLTFGGGT |
| | KVEIK |
| 28 | EVQLLESGGG LVQPGKSLRL SCAVSGFTFS TYGMNWVRQA |
| SEQ ID NO: 18 of | PGKGLEWVSS ISGTGRTTYH ADSVQGRFTV SRDNSKNILY |
| WO2012064682 | LQMNSLRADD TAVYFCTKER GDYYYGVFDY WGQGTLVTVS S |
| 29 | DIQMTQSPST LSASVGDRVT ITCRASQTIS SWLAWYQQTP |
| SEQ ID NO:26 of | EKAPKLLIYA ASNLQSGVPS RFSGSGSGTE FTLTISSLQP |
| WO2012064682 | DDFATYYCQQ YHRSWTFGQG TKVEIT |
| 30 | QVQLVQSGAE VKKPGASVKV SCKASGYTFT SYDINWVRQA |
| C320-168 huIgG1- | PGQGLEWMGW LNPNSGNTGY AQKFQGRVTM TRNTSISTAY |
| EFN | MELSSLRSED TAVYYCAREV PETAAFEYWG QGTLVTVSSA |
| of WO2013044298 | STKGPSVFPL APSSKSTSGG TAALGCLVKD YFPEPVTVSW |
| | NSGALTSGVH TFPAVLQSSG LYSLSSVVTV PSSSLGTQTY |
| | ICNVNHKPSN TKVDKKVEPK SCDKTHTCPP CPAPEAAGAP |
| | SVFLFPPKPK DTLMISRTPE VTCVVVDVSH EDPEVKFNWY |
| | VDGVEVHNAK TKPREEQYNS TYRVVSVLTV LHQDWLNGKE |
| | YKCKVSNKAL PAPIEKTISK AKGQPREPQV YTLPPSREEM |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| 31 | QSVLTQPPSV SGAPGQRVTI SCTSSSSDIG AGLGVHWYQQ |
| C320-168 | LPGTAPKLLI YGYYNRPSGV PDRFSGSKSG TSASLTITGL |
| huLambda | LPEDEGDYYC QSYDGTLSAL FGGGTKLTVL GQPKAAPSVT |
| of WO2013044298 | LFPPSSEELQ ANKATLVCLI SDFYPGAVTV AWKADSSPVK |
| | AGVETTTPSK QSNNKYAASS YLSLTPEQWK SHRSYSCQVT |
| | HEGSTVEKTV APTECS |
| 32 | QVQLVQSGAE VKKPGASVKV SCKASGYTFT SYDINWVRQA |
| VH C320-179 of | PGQGLEWMGW LNPNSGNTGY AQKFQGRVTM TADRSTSTAY |
| WO2013044298 | MELSSLRSED TAVYYCAREV PETAAFEYWG QGTLVTVSS |
| 33 | QSVLTQPPSV SGAPGQRVTI SCTSSSSDIG AGLGVHWYQQ |
| VL C320-179 of | LPGTAPKLLI EGYYNRPSGV PDRFSGSKSG TSASLTITGL |
| WO2013044298 | LPEDEGDYYC QSYDGTLSAL FGGGTKLTVLG |

The antibodies as described herein can be made by any method known in the art. For the production of hybridoma cell lines, the route and schedule of immunization of the host animal are generally in keeping with established and conventional techniques for antibody stimulation and production, as further described herein. General techniques for production of human and mouse antibodies are known in the art and/or are described herein.

It is contemplated that any mammalian subject including humans or antibody producing cells therefrom can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies.

Hybridomas that produce antibodies used for the present invention may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing the antibody target (e.g., PD-1), a human target protein (e.g., PD-1), or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups, can yield a population of antibodies (e.g., monoclonal antibodies).

If desired, the antibody (monoclonal or polyclonal) of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., J. Immunol. Methods 329, 112, 2008; U.S. Pat. No. 7,314,622.

In some embodiments, antibodies may be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

In some embodiments, antibodies as described herein are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art, some of which are described below and in the Examples.

### Polynucleotides, Vectors, and Host Cells

The invention also provides polynucleotides encoding any of the anti-TL1A antibodies as described herein. Polynucleotides can be made and expressed by procedures known in the art.

In another aspect, the invention provides compositions (such as a pharmaceutical compositions) comprising any of the polynucleotides of the invention, for use in one or more methods of the invention. In some embodiments, the composition comprises an expression vector comprising a polynucleotide encoding any of the anti-TL1A antibodies described herein, for use in one or more methods of the invention.

In another aspect, provided is an isolated cell line that produces the anti-TL1A antibodies as described herein for use in one or more methods of the invention.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an antibody or a fragment thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a fragment thereof.

### Compositions

The invention also provides pharmaceutical compositions comprising an effective amount of an anti-TL1A antibody as described herein, and such pharmaceutical compositions for use in methods of treatment as described herein. Examples of such compositions, as well as how to formulate, are also described herein. It is understood that the compositions can comprise more than one anti-TL1A antibody.

The composition used in the present invention can further comprise pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable excipients are further described herein.

The anti-TL1A antibody, and compositions thereof, can also be used in conjunction with, or administered separately, simultaneously, or sequentially with other agents that serve to enhance and/or complement the effectiveness of the agents.

### Formulations

Therapeutic formulations of the anti-TL1A antibody used in accordance with the present invention are prepared for storage by mixing the protein having the desired degree of purity with optional pharmaceutically acceptable carriers, excipients or stabilizers (Remington, The Science and Practice of Pharmacy 20th Ed. Mack Publishing, 2000), in the form of lyophilized formulations or aqueous solutions. Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the anti-TL1A antibody are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic anti-TL1A antibody compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an anti-TL1A antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulised by use of gases. Nebulised solutions may be breathed directly from the nebulising device or the nebulising device may be attached to a face mask, tent or intermittent positive pressure breathing machine. Solution, suspension or powder compositions may be administered, preferably orally or nasally, from devices which deliver the formulation in an appropriate manner.

In embodiments that refer to a method of treating IBD as described herein, such embodiments are also further embodiments of an anti-TL1A antibody for use in that treatment, or alternatively of the use of an anti-TL1A antibody in the manufacture of a medicament for use in that treatment.

### Methods of Treatment

In some aspects, the invention relates to a method for treating inflammatory bowel disease (IBD) in a patient, the method comprising administering to the patient an anti-TNF-like ligand 1A (TL1A) antibody in an induction dosing regimen sufficient to improve signs and symptoms of IBD by at least 12 weeks after the start of treatment with the anti-TL1A antibody, said induction dosing regimen comprising a plurality of individual induction doses, wherein the method further comprises administering to the patient a subsequent maintenance dosing regimen after completion of the induction dosing regimen, said maintenance dosing regimen comprising a plurality of individual maintenance doses separated from each other by at least 2 weeks. One or more of the individual maintenance doses may be administered at least 4, 8, 12, 16, or 24 weeks apart.

In some aspects, the invention provides a method for treating inflammatory bowel disease (IBD) in a patient, the method comprising administering to the patient an anti-TNF-like ligand 1A (TL1A) antibody in a therapeutic dosing regimen sufficient to improve signs and symptoms of IBD by at least 12 weeks after the start of treatment with the anti-TL1A antibody, said induction dosing regimen comprising a plurality of individual induction doses, wherein the method further comprises administering to the patient a subsequent maintenance dosing regimen after completion of the induction dosing regimen, said maintenance dosing regimen comprising a plurality of individual maintenance doses separated from each other by at least 1, 2, 3, 4, or 6 months.

The time interval between each individual maintenance dose may be the same. The individual maintenance doses may be about 100% of the individual induction dose, or they may be no more than about 75% of the individual induction doses, no more than about 50% of the individual induction doses, no more than about 40% of the individual induction doses, no more than about 25% of the individual induction doses, or no more than about 20% of the individual induction doses. In some aspects, one or more of the individual maintenance dose is selected from the group consisting of 500, 450, 400, 350, 300, 250, 200, 150, 100, and 50 mg.

One or more of the individual induction dose may be about 500 mg via intravenous injection. One or more of the individual induction doses may be separated from each other by 2 weeks.

In some aspects, the invention provides a method for treating inflammatory bowel disease (IBD) in a patient sufficient to improve signs and symptoms of IBD, the method comprising administering to the patient an anti-TNF-like ligand 1A (TL1A) antibody in an induction dosing regimen, said induction dosing regimen comprising a plurality of individual induction doses of 500 mg every 2 weeks via intravenous injection.

The induction dosing regimen may be continued for at least 12 weeks.

The maintenance dosing regimen may be maintained for at least 2, 3, 4, or 6 months.

Following the induction dosing regimen the patient may experience an improvement in signs and symptoms of IBD characterized by a clinical response. The term “clinical response” is a decrease from baseline of at least 3 points in total Mayo score with at least 30% change, accompanied by at least one-point decrease or absolute score of 0 or 1 in rectal bleeding subscore.

The abbreviation “Mayo” means the Mayo Scoring System for Assessment of Ulcerative Colitis Activity. “Adaptive Mayo Score” refers to the Adaptive Mayo Score system which has 3 subscores of the Mayo Score ranging from 0 to 9 without PGA subscore.

Following the induction dosing regimen the patient may experience an improvement in signs and symptoms of IBD characterized by an endoscopic response. The term “endoscopic response” refers to a Mayo endoscopy subscore 0 or 1.

Following the induction dosing regimen the may patient experience an improvement in signs and symptoms of IBD characterized by a clinical remission. The term “clinical remission” is based on 12-point total Mayo score: total Mayo score with no individual subscore >1.

Following the induction dosing regimen the patient may experience an improvement in signs and symptoms of IBD characterized by an endoscopic remission. The term “endoscopic remission” refers to a Mayo endoscopy subscore 0.

Following the induction dosing regimen the patient may experience an improvement in signs and symptoms of IBD characterized by a deep remission. The term “deep remission” refers to a total Mayo score of 2 points or lower, with no individual subscore exceeding 1 point and a 0 on both endoscopic and rectal bleeding subscore.

Following the induction dosing regimen the patient may experience an improvement in signs and symptoms of IBD characterized by a symptomatic remission. The term “symptomatic remission” refers to a total Mayo score of 2 points or lower, with no individual subscore exceeding 1 point, and both rectal bleeding and stool frequency subscores of 0.

Following the induction dosing regimen the patient may experience an improvement in signs and symptoms of IBD characterized by an endoscopic improvement. The term “endoscopic improvement” (“EI”) refers to a decrease of ≥1 point in Mayo endoscopy subscore or an absolute endoscopy score of ≤1.

Following the induction dosing regimen the patient may experience an improvement in signs and symptoms of IBD that are maintained while the patient receives the maintenance dosing regimen.

In some aspects of the invention, the induction dosing regimen with the anti-TL1A antibody effectively improves signs and symptoms of IBD by at least 14 weeks after starting of treatment with the anti-TL1A antibody. These improvement in signs and symptoms of IBD may be characterized by an improvement in the Mayo endoscopic subscore. The reduction of the patient's Mayo endoscopic subscore may be by at least 1, 2, or 3 or more integers.

The improvement in signs and symptoms of IBD may be characterized by the patient having a Mayo endoscopic subscore of 0 or 1, 2, or 3. The improvement in signs and symptoms of IBD may be characterized by the patient having a total Mayo score of 0, 1, 2, or 3. The improvement in signs and symptoms of IBD may be characterized by the patient having a Robarts Histopathology Index (RHI) of less than 5. The improvement in signs and symptoms of IBD may be characterized by the patient having a Geboes Index of less than 3.2.

The improvement in signs and symptoms of IBD may be maintained during the maintenance dosing regimen for at least 2, 3, 4, 6, or 12 months.

In some aspects, the invention relates to method for treating inflammatory bowel disease (IBD) in a patient, the method comprising administering to the patient an anti-TNF-like ligand 1A (TL1A) antibody in an induction dosing regimen sufficient to improve signs and symptoms of IBD by at least 12 weeks after the start of treatment with the anti-TL1A antibody, said induction dosing regimen comprising 6 individual induction doses each of 500 mg administered 2 weeks apart, wherein the method further comprises administering to the patient a subsequent maintenance dosing regimen after completion of the induction dosing regimen, said maintenance dosing regimen comprising a plurality of individual maintenance doses, each individual maintenance dose being no more than 75% of the individual induction dose, and wherein each individual maintenance dose is separated from each other by at least 4 weeks.

In some aspects, the patient was previously treated with corticosteroids prior to administering the anti-TL1A antibody. In some aspects the patient was previously treated with one or more treatments selected from the group consisting of tumor necrosis factor inhibitors, anti-integrins, azathioprine, 6-mercaptopurine, and methotrexate.

In some aspects the patient shows a reduction of fecal calprotectin from baseline of at least 50% from week 2 to week 26 of treatment. In some aspects the patient shows a reduction of fecal calprotectin from baseline of at least 60% from week 2 to week 26 treatment. In some aspects the patient shows a reduction of hsCRP from baseline from week 2 to week 26 of treatment.

In some aspects the IBD is ulcerative colitis (UC). In some aspects, the patient has moderate to severe ulcerative colitis. The term “moderate to severe ulcerative colitis” is defined as having an Adapted Mayo score of 5 to 9, with an endoscopy subscore of 2 or 3.

In some aspects of the invention, the anti-TL1A antibody comprises three CDRs from the variable heavy chain region having the sequence shown in SEQ ID NO: 1 and three CDRs from the variable light chain region having the sequence shown in SEQ ID NO: 2.

In some aspects of the invention, the anti-TL1A antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

In some aspects of the invention, the anti-TL1A antibody comprises a variable heavy chain region having the sequence shown in SEQ ID NO: 1 and a variable light chain region having the sequence shown in SEQ ID NO: 2.

In some aspects of the invention, the anti-TL1A antibody comprises a heavy chain having the sequence shown in SEQ ID NO: 9 and a light chain having the sequence shown in SEQ ID NO: 10, wherein the C-terminal lysine (K) of the heavy chain amino acid sequence of SEQ ID NO: 9 is optional.

In some aspects of the invention, the anti-TL1A antibody comprises a VH encoded by the nucleic acid sequence of the insert of the vector deposited as 1D1 1.31 VH having ATCC accession number PTA-120639 and a VL encoded by the nucleic acid sequence of the insert of the vector deposited as 1D11.31 VL having ATCC accession number PTA-120640.

In some aspects of the invention, the anti-TL1A antibody competes for binding with an anti-TL1A antibody comprising a variable heavy chain region having the sequence shown in SEQ ID NO: 1 and a variable light chain region having the sequence shown in SEQ ID NO: 2.

In some aspects of the invention, the anti-TL1A antibody competes for binding with an antibody comprising a VH encoded by the nucleic acid sequence of the insert of the vector deposited as 1D11.31 VH having ATCC accession number PTA-120639 and a VL encoded by the nucleic acid sequence of the insert of the vector deposited as 1D11.31 VL having ATCC accession number PTA-120640.

In some aspects of the invention, the anti-TL1A antibody comprises sequence pairs selected from the group consisting of SEQ ID NO:4 and 11; SEQ ID NO:4 and 12; SEQ ID NO:4 and 13; SEQ ID NO:4 and 14; SEQ ID NO:4 and 15; SEQ ID NO:4 and 16; SEQ ID NO:4 and 17; SEQ ID NO:4 and 18; SEQ ID NO:4 and 19; SEQ ID NO:20 and 24; SEQ ID NO:21 and 25; SEQ ID NO:22 and 26; SEQ ID NO:23 and 27; SEQ ID NO:28 and 29; SEQ ID NO:30 and 31; and SEQ ID NO:30 and 31.

In some aspects of the invention, the method further comprises the steps of:
a) determining the expression level of one or more candidate genes in a sample from the patient,
b) identifying that the sample contains an abnormal expression level of the one of more candidate gene,
c) administering the induction dosing regimen or individual induction dose of the anti-TL1A antibody to a patient.

Determining whether the patient has an abnormal expression level of a one or more candidate gene may be by obtaining or having obtained a sample from the patient. The sample may be a tissue sample. The sample may be a tissue sample from a site of IBD inflammation. The sample may be a peripheral blood sample. The sample may be an intestinal biopsy sample.

The method may further comprise performing or having performed an assay on the sample to determine if the patient expresses abnormal levels of the one or more candidate gene.

In some aspects, if the sample contains abnormal levels of the one or more candidate gene then the method provides for the further step of administering an induction dosing regimen or induction dose of the anti-TL1A antibody to the patient.

In some aspects, the risk of the patient being non-responsive to an induction dosing regimen or individual induction dose of anti-TL1A antibody is lower in a patient with abnormal levels of the one or more candidate gene.

In some aspects, the invention provides a method for treating inflammatory bowel disease (IBD) in a patient, the method comprising the steps of:
a) determining the expression level of one or more candidate genes in a sample from the patient,
b) identifying that the sample contains an abnormal expression level of the one of more candidate gene,
c) administering an induction dosing regimen or individual induction dose of an anti-TNF-like ligand 1A (TL1A) antibody to the patient.

The one or more candidate genes may be selected from the group consisting of IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L. The one or more candidate genes may be selected from the group consisting of IL-1B, IL-23A, IFNG, IL-12RB1, IL-21R, IRF4, and BATF. The one or more candidate genes may be selected from the group consisting of CD80/86, HLA-DRB5/DQB1/DRB1, HLA-DRA, CD40, and ICOS. The one or more candidate genes may be selected from the group consisting of MMP3, MMP7, MMP10 and CHI3L.

The one or more candidate genes may be selected from the group consisting of SOWAHB, COLCA2, TBX20, FRZB, HOXB5, NET1, FOXD2, DESI1, PARK2, PKDREJ, IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L.

The one or more candidate genes may be selected from the group consisting of SOWAHB, COLCA2, TBX20, FRZB, HOXB5, NET1, FOXD2, DESI1, PARK2, and PKDREJ. The one or more candidate genes may comprise SOWAHB. The one or more candidate genes may comprise SOWAHB, and at least one or more candidate genes selected from the group consisting of COLCA2, TBX20, FRZB, HOXB5, NET1, FOXD2, DESI1, PARK2, and PKDREJ. The one or more candidate genes may comprises SOWHAB and COLCA2, and at least one or more candidate genes selected from the group consisting of SOWAHB, COLCA2, TBX20, FRZB, HOXB5, NET1, FOXD2, DESI1, PARK2, and PKDREJ. The one or more candidate genes may comprise SOWAHB, COLCA2, and TBX20 and at least one or more candidate genes selected from the group consisting of FRZB, HOXB5, NET1, FOXD2, DESI1, PARK2, and PKDREJ.

In some aspects, the method provide 2, or 3, or 4, or 5, or 6, or 7, or 8 or 9, or 10 of the candidate genes selected from the group consisting of SOWAHB, COLCA2, TBX20, FRZB, HOXB5, NET1, FOXD2, DESI1, PARK2, and PKDREJ.

The abnormal expression level of the one or more candidate gene may be based on the one or more candidate gene's level of mRNA or expressed protein. The abnormal expression level of the one or more candidate gene may be based on the one or more candidate gene's mRNA levels.

The expression level of the one or more candidate gene may be compared against a baseline expression level which is based on the expression level of the one or more candidate gene for a healthy individual who is not suffering from IBD or UC.

The expression level of the one or more candidate gene may be compared against a baseline expression level which is based on an estimated expression level for individuals who are non-responsive to anti-TL1A antibody treatment.

The abnormal expression level of the one or more candidate gene may be at least 50% greater or lesser from the baseline level. The abnormal expression level of the one or more candidate gene is at least 2-fold, 5-fold, 10-fold, 50-fold, 100-fold, 500-fold, or 1000-fold greater or lesser from the baseline level.

In some aspects, when the one or more of the candidate genes is selected from the group consisting of SOWAHB, COLCA2, FRZB, HOXB5, NET1, FOXD2, PARK2, and PKDREJ, then the abnormal expression level may be an elevated level. In some aspects, when the one or more of the candidate genes is selected from the group consisting of TBX20 and DESI1, then the abnormal expression level is a decreased level.

In some aspects, the invention a method for treating a patient with an anti-TNF-like ligand 1A (TL1A) antibody, wherein the patient is suffering from inflammatory bowel disease (IBD), the method comprising the steps of:
a) determining whether the patient is a haplotype A, B or C for TNFSF15 by obtaining or having obtained a biological sample from the patient;
b) performing or having performed a genotyping assay on the biological sample to determine if the patient is of haplotype A, B or C for TNFSF15;
   wherein the risk of the patient being non-responsive to the induction dosing regimen or individual induction dose of anti-TL1A antibody is lower in a patient of haplotype A or haplotype C than in a patient of haplotype B; and further, wherein one or both of
   (a) if the patient is of haplotype B for TNFSF15 then administering a maintenance dosage regimen of the anti-TL1A antibody to the patient that provides an increased individual maintenance dose relative to the individual maintenance dose provided to patients of haplotype A or C; and
   (b) wherein if the patient is of haplotype B for TNFSF15 then administering a maintenance dosage regimen of the anti-TL1A antibody to the patient that provides a decreased time interval between the individual maintenance doses relative to the time intervals between individual maintenance doses provided to patients of haplotype A or C.

In some aspects, the invention comprises the steps of:
a) determining the level of one or more candidate bacterial strains in a stool sample from the patient,
b) identifying that the stool sample contains an elevated level of the one of more candidate bacterial strains,
c) administering the induction dose of the anti-TL1A antibody to a patient.

In some aspects, the candidate bacterial strain is selected from the group consisting of Streptococcus salivarius, Streptococcus. parasanguinis, and Haemophilus parainfluenzae.

In some aspects, the invention comprises the steps of:
a) determining the level of one or more candidate bacterial strains in a stool sample from the patient,
b) identifying that the stool sample contains a decreased level of the one of more candidate bacterial strains,
c) administering the induction dose of the anti-TL1A antibody to a patient.

In some aspects, the candidate bacterial strain is selected from the group consisting of Ruminococcus albus, Ruminococcus callidus, Ruminococcus Ruminococcus gnavus, and Bifidobacterium bifidum.

In some aspects, the level of the one or more candidate bacterial strains is compared against a baseline bacterial level which is based on the level of the one or more candidate bacterial strain for a healthy individual who is not suffering from IBD or UC. In some aspects, the level of the one or more candidate bacterial strains is compared against a baseline bacterial level which is based on an estimated level of those candidate bacterial strains for individuals who are non-responsive to anti-TL1A antibody treatment.

In some aspects, the level of the one or more candidate bacterial strains is at least greater or lesser from the baseline bacterial level by at least 50%, 2-fold, 5-fold, 10-fold, 20-fold, 50-fold, 100-fold, 500-fold, or 1000-fold.

In some aspects, the invention further comprises treatment with an IL-23 antagonist.

The invention provides a method for identifying a patient having inflammatory bowel disease as being likely to benefit from initial or continued treatment with anti-TL1A antibody treatment, and optionally treating said patient, wherein said method comprises:
(a) identifying a patient as containing an abnormal level of one or more candidate genes, selected from the group consisting of SOWAHB, COLCA2, TBX20, FRZB, HOXB5, NET1, FOXD2, DESI1, PARK2, PKDREJ, IL-1B, IL-23A, IFNG, IL-12RB1, IL-21R, IRF4, BATF, CD80/86, HLA-DRB5/DQB1/DRB1, HLA-DRA, CD40, ICOS, MMP3, MMP7, MMP10, and CHI3L;
(b) administering or having administered to said patient an anti-TL1A antibody under conditions wherein one or more selected from the group consisting of inflammatory macrophages, TH17, ILC3, OX40, OX40L, IFNγ, ILC2, IL-13, MMP, tissue remodeling, fibrosis, the intestinal population of S. salivarius, the intestinal population of S. parasanguinis, and the intestinal population of H. parainfluenzae in said patient is reduced after said administering.

### Kits

The invention also provides kits comprising any or all of the anti-TL1A antibodies described herein. Kits of the invention include one or more containers comprising an anti-TL1A antibody described herein and instructions for use in accordance with any of the methods of the invention described herein. Generally, these instructions comprise a description of administration of the anti-TL1A antibody for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of an anti-TL1A antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-TL1A antibody. The container may further comprise a second pharmaceutically active agent.

Kits may optionally provide additional components such as buffers and interpretive information. Normally, the kit comprises a container and a label or package insert(s) on or associated with the container.

### EXAMPLES

### Example 1

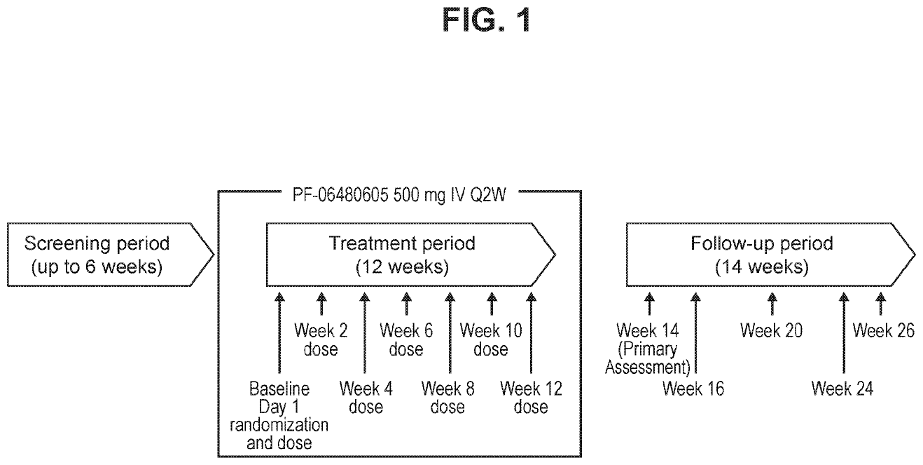

A Phase 2a, single-arm study described herein (TUSCANY; NCT02840721), employed a Simon's 2-stage design (Simon R. Control Clin Trials 1989; 10:1-10). Participants with moderately to severely active UC were enrolled in the first stage to receive a 60-minute IV infusion of 500 mg PF-06480605 Q2W for 7 doses. The induction period took place between baseline and Week 12, after a screening period of up to 6 weeks, with endoscopic assessment performed at Week 14, 2 weeks after the last dose (FIG. 1). The primary efficacy endpoint was assessed for futility at the end of the first stage under Simon's 2-stage design decision criteria in 12 evaluable participants with colonoscopy. If participants had achieved endoscopic improvement (EI), and no participant had achieved endoscopic remission, then the study would be stopped for futility. If futility criteria were not met, enrolment of additional participants in the second stage would continue.

### Mayo Score for Ulcerative Colitis Activity

The Mayo Score is a tool designed to measure disease activity for UC. The Mayo scoring system consists of 4 subscores, each graded 0 to 3 with the higher score indicating more severe disease activity (See below and Section 10.9.3). The total Mayo score is a summary of all 4 subscores ranging from 0 to 12 points.
Stool frequency (Subscore 0-3).
Rectal bleeding (Subscore 0-3).
Findings on endoscopy (Subscore 0-3).
Physician's Global Assessment (Subscore 0-3).

Calculation of the Mayo Score requires an assessment of the participant's stool frequency and any amount of blood in the stool. The Mayo scores will be calculated based on the participant's stool diary most recently recorded 3 valid and consecutive days closest to the study visit. Investigator sites will be trained on the diary usage and will train participants on use of the diary. Diary data entered by the participant will be reviewed by the site at each visit.

If there are missing stool diary data, the average will be taken from the 3 most recently available days reported within 5 days close to (and prior to if it is a baseline visit) study visit for calculation of Mayo score. Invalid days for Mayo score calculation are dates for bowel preparation, endoscopy and 1 day after endoscopic procedure.

If there only 2 available valid days reported within the 5 days close to (and prior to if it is a baseline visit) the study visit, the average will be taken from the limited available data unless there is no diary data reported within 5 days. In this case, stool frequency and rectal bleeding subscores will be considered as missing. The rectal bleeding subscore will be rounded up if the average is between >0 to <1.

### Participants

Male and female participants ≥18 and ≤75 years of age (or ≥19 and ≤75 years of age if enrolled in the Republic of Korea) were eligible to participate if they met the following key inclusion criteria: A diagnosis of moderately to severely active UC for ≥4 months, defined by a screening colonoscopy in which the total Mayo score was ≥6, the rectal bleeding score was ≥1, and endoscopic subscore was ≥2; active disease beyond the rectum identified at the screening colonoscopy, and inadequate response, loss of response or intolerance to ≥1 conventional therapy for UC, namely corticosteroids, immunosuppressants, immunomodulators, or anti-integrins. Participants were ineligible to participate if they met any of the following key exclusion criteria: A diagnosis of indeterminate colitis, ischemic colitis, radiation colitis, diverticular disease, microscopic colitis or Crohn's disease; an imminent need, or prescheduled appointment for, surgery that would take place during the study; the presence of colonic dysplasia, neoplasia, toxic megacolon, primary sclerosing cholangitis or colonic stricture; a history of colonic or small bowel stoma, obstruction or resection, or a transplanted organ; the presence of active enteric infections, human immunodeficiency virus infection, tuberculosis infection or any other significant concurrent medical condition; abnormal laboratory parameters at screening, or participants who were receiving, or expected to receive, >9 mg/day of oral budesonide or >20 mg/day of prednisone or equivalent oral systemic corticosteroid dose within 2 weeks prior to baseline; IV, intramuscular (parenteral), or topical (rectal) treatment of 5-ASA or corticosteroid enemas/suppositories within 2 weeks prior to baseline; infliximab, adalimumab or golimumab within 8 weeks prior to baseline; or vedolizumab 12 weeks prior to baseline.

### Safety Assessments

Investigating the safety and tolerability of PF-06480605 was the primary objective of this study. Treatment-emergent adverse events (TEAEs), including serious AEs (SAEs) and treatment-related TEAEs, were reported according to the Medical Dictionary for Regulatory Activities (MedDRA) coding dictionary version 21.0. An SAE was any TEAE that resulted in death, was life-threatening, required hospitalization, or resulted in disability or congenital abnormality. Laboratory parameters, including aspartate transaminase, alanine aminotransferase, and total bilirubin were also monitored for signs of drug-induced liver injury, and vital signs were monitored for other abnormalities.

### Efficacy Assessments

Treatment efficacy was assessed based on Mayo Score, (Schroeder et al. N Engl J Med 1987; 317:1625-1629) a tool to measure disease activity in UC. The Mayo scoring system ranges from 0 to 12 points and comprises 4 subscores (stool frequency, rectal bleeding, findings on endoscopy, and physician's global assessment), each graded 0 to 3, where higher scores correspond to more severe disease activity. The primary efficacy endpoint was EI at Week 14, defined by a Mayo endoscopic subscore of 0 or 1, without friability. To ensure objective and consistent assessment of the primary endpoint, the Mayo endoscopic subscore was determined through blinded, centrally read colonoscopy images with built-in adjudications. Secondary efficacy endpoints were remission (total Mayo score with no individual subscore >1) and endoscopic remission (Mayo endoscopic subscore of 0) at Week 14.

Exploratory endpoints included clinical remission at Week 14 (defined as a Mayo endoscopic subscore of 0 or 1, without friability, with stool frequency and rectal bleeding subscores of 0), change from baseline in partial Mayo score, clinical response (defined as a decrease from baseline in total Mayo score by at least 3 points and at least 30%, with a decrease in rectal bleeding subscore of at least 1 point or an absolute subscore of 0 or 1) at Week 14, and minimal histologic activity (defined as a Geboes Index [GI] of ≤3.2 or Robarts Histopathology Index [RHI] of ≤5) (Sands B E, Peyrin-Biroulet L, Loftus E V, Jr., et al. Vedolizumab versus adalimumab for moderate-to-severe ulcerative colitis. N Engl J Med 2019; 381:1215-1226) and histologic remission (defined as GI ≤3.0 or RHI ≤6) at Week 14.

### Pharmacokinetic and Immunogenicity Assessments

Other secondary endpoints included analysis of pharmacokinetics (PK), biomarkers including soluble TL1A (sTL1A) concentration, fecal calprotectin, and high-sensitivity C reactive protein (hsCRP). Incidence of anti-drug antibodies (ADA) and neutralizing antibodies (NAb) was determined using ligand binding and cell-based immunogenicity assays, respectively.

### Statistical Analyses

This study was designed based on a Simon's 2-stage design, testing the proportion of participants achieving EI at Week 14 (p) with the hypotheses H₀ (null): p ≤6% versus H₁ (alternative): p ≥41%, where 6% was the observed placebo EI rate in anti-TNF-experienced participants based on a reanalysis of two Phase 3 induction studies of tofacitinib 10 mg²¹ (OCTAVE Induction 1 [NCT01465763]; OCTAVE Induction 2 [NCT01458951]), and 41% corresponds to the effect of a transformational drug. At the end of the first stage of the design, it was planned to have 12 evaluable participants for futility analysis. If futility criteria were not met, enrollment continued until at least 36 evaluable participants completed the study. The primary efficacy endpoint, EI at Week 14, was analyzed based on the uniformly minimum-variance unbiased estimator (UMVUE) method (Jung and Kim 2004, Stat Med; 23:881-896; Koyama and Chen 2008, Stat Med; 27:3145-3154) and the maximum likelihood estimator (MLE) method using data from the per-protocol (PP) population, which comprised participants who were eligible for enrollment, with at least 6/7 planned doses received, and a final colonoscopy at Week 14. For the testing of statistical significance, a P-value of <0.05 was required. All study endpoints were summarized descriptively.

### Ethics

The final protocol and any amendments were reviewed and approved by Institutional Review Boards/Independent Ethics Committees at each participating center. The study was conducted in compliance with the Declaration of Helsinki and with all International Council for Harmonisation Good Clinical Practice guidelines. All participants provided written, informed consent to participate. All authors had access to the study data and reviewed and approved the final manuscript for publication.

### Results

### Participants

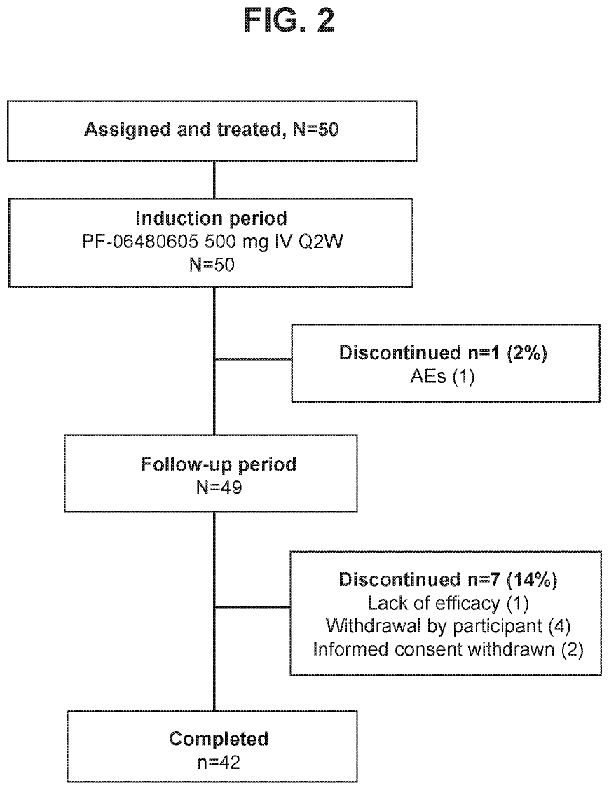

A total of 50 participants received PF-06480605, of whom 42 completed the study to follow-up (FIG. 2). Participant demographics and baseline disease characteristics are summarized in Table 2. A majority of participants were male (28/50, 56.0%), and most were white (48/50, 96.0%). The mean age was 40.0 years. The most common forms of UC at baseline were pancolitis (24/50, 48.0%) and left-sided colitis (16/50, 32.0%).

TABLE 2
Participant demographics and baseline disease characteristics
(safety analysis set)

| | PF-06480605 |
|---|---|
| | 500 mg IV |
| | Q2W N = 50 |
| Gender, n (%) | |
| Male | 28 (56.0) |
| Female | 22 (44.0) |
| Mean age, years (SD) | 40.0 (14.5) |
| Race, n (%) | |
| White | 48 (96.0) |
| Asian | 2 (4.0) |
| Mean weight, kg (SD) | 70.1 (14.8) |
| Mean BMI, kg/m² (SD) | 23.6 (4.4) |
| Mean height, cm (SD) | 172.2 (10.4) |
| Extent of disease, n (%) | |
| Proctitis | 0 (0%) |
| Procto-sigmoiditis | 7 (14.0%) |
| Left-sided colitis | 16 (32.0%) |
| Extensive colitis | 3 (6.0%) |
| Pancolitis | 24 (48.0%) |

BMI, body mass index; IV, intravenous; n, number of participants in the specified category; N, number of participants overall (safety population); Q2W, every 2 weeks; SD, standard deviation.

At baseline, most participants (46, 92.0%) had received corticosteroids for the treatment of UC. Prior treatment with biologic therapies, specifically TNFi and anti-integrins, was reported in 36 (72.0%) and 28 participants (56.0%), respectively, and a further 33 (66.0%), 10 (20.0%), and 4 (8.0%) participants had received azathioprine, 6-mercaptopurine, and methotrexate, respectively.

### Safety

The mean duration of treatment was 82.6 days, corresponding to 500 mg IV PF-06480605 Q2W for a maximum of 7 doses. The majority of participants (35/50, 70.0%) had a PF-06480605 exposure duration of 85-98 days. Most participants (46/50, 92.0%) received 7 doses of PF-06480605. Six, 5, and 1 dose(s) were received by 2, 1, and 1 participant(s), respectively. In total, 33 participants reported 109 all-causality TEAEs. Of these, 18 were treatment-related, which were experienced by 8 participants. Three participants experienced 4 SAEs, which were UC disease flare and subsequent peritonitis in 1 participant (not treatment-related), UC disease flare in 1 participant (not treatment-related), and alopecia areata in 1 participant (considered treatment-related by the investigator). Of these 3 participants, 2 discontinued PF-06480605 due to SAEs of unrelated UC and treatment-related alopecia areata (1 participant each) but continued the study. One other participant permanently discontinued the study due to a TEAE of UC (not treatment-related).

Table 3 shows a summary of all-causality and treatment-related TEAEs, and TEAEs by MedDRA system organ class (SOC). Other than UC, the most common TEAEs by preferred term were arthralgia in 6 participants (12.0%), in 1 of whom (2.0%) the arthralgia was treatment-related. The following occurred in 3 participants (6.0%) each: abdominal pain (1 treatment-related [2.0%]), nausea (1 treatment-related [2.0%]), nasopharyngitis (none treatment-related), pharyngitis (none treatment-related), back pain (1 treatment-related [2.0%]) and alopecia areata (1 treatment-related [2.0%]). There were no deaths or malignancies and no clinically significant findings for vital signs or laboratory parameters.

TABLE 3
Summary of all-causality and treatment-related TEAEs, and TEAEs by
SOC (safety analysis set)

| | All-causality | T reatment-related |
|---|---|---|
| | TEAEs | TEAEs |
| | in participants | in participants |
| | receiving | receiving |
| | PF-06480605 | PF-06480605 |
| | 500 mg IV Q2W | 500 mg IV Q2W |
| | n (%) | n (%) |
| Participants evaluable for AEs | 50 (100.0) | 50 (100.0) |
| Participants with AEs | 33 (66.0) | 8 (16.0) |
| Participants with SAEs | 3^{a} (6.0) | 1 (2.0) |
| Participants discontinued due | 3^{b} (6.0) | 1 (2.0) |
| to AEs | | |
| TEAEs by SOC | | |
| Blood and lymphatic system | 2 (4.0) | 0 (0.0) |
| disorders | | |
| Cardiac disorders | 2 (4.0) | 0 (0.0) |
| Ear and labyrinth disorders | 2 (4.0) | 1 (2.0) |
| Endocrine disorders | 1 (2.0) | 0 (0.0) |
| Gastrointestinal disorders | 14 (28.0) | 2 (4.0) |
| General disorders and | 7 (14.0) | 2 (4.0) |
| administration-site conditions | | |
| Infections and infestations | 13 (26.0) | 0 (0.0) |
| Injury, poisoning, and | 5 (10.0) | 0 (0.0) |
| procedural complications | | |
| Investigations | 6 (12.0) | 1 (2.0) |
| Metabolism and nutrition | 1 (2.0) | 0 (0.0) |
| disorders | | |
| Musculoskeletal and | 9 (18.0) | 2 (4.0) |
| connective tissue disorders | | |
| Neoplasms benign, malignant, | 2 (4.0) | 1 (2.0) |
| and unspecified (including | | |
| cysts and polyps) | | |
| Nervous system disorders | 1 (2.0) | 1 (2.0) |
| Respiratory, thoracic, and | 3 (6.0) | 1 (2.0) |
| mediastinal disorders | | |
| Skin and subcutaneous tissue | 7 (14.0) | 2 (4.0) |
| disorders | | |
| Vascular disorders | 4 (8.0) | 2 (4.0) |

^{a}Three participants experienced 4 SAEs: UC and peritonitis in 1 participant (not treatment-related), UC in 1 participant (not treatment-related), and alopecia areata in 1 participant (treatment-related).
^{b}One participant permanently discontinued from the study due to UC (not treatment-related);
2 participants discontinued treatment but continued the study: 1 due to UC (not treatment-related) and 1 due to alopecia areata (treatment-related).
AE, adverse event; IV, intravenous; n, number of participants in the specified category;
Q2W, every 2 weeks; SAE, serious adverse event; SOC, system organ class;
TEAE, treatment-emergent adverse event; UC, ulcerative colitis.

### Efficacy

EI at Week 14 in the PP population was observed in 38.2% of cases (based on UMVUE) and ranged from 35.0-60.0% in participants with baseline endoscopic subscores of 2, 2/3, and 3 (based on MLE; Table 5). This result was statistically significant, and the null hypothesis of 6% was rejected with a P-value of <0.001 (UMVUE). Four sensitivity analyses yielded similar results to the MLE analyses for the primary endpoint of EI at Week 14.

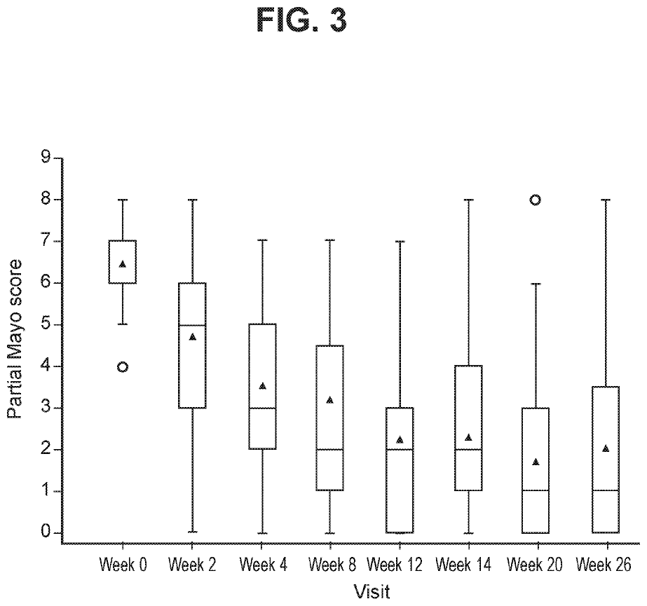

The proportion of participants achieving remission and endoscopic remission at Week 14 (secondary endpoints) was 24.0% and 10.0%, respectively, using the MLE method in the full analysis set (FAS) with non-responder imputation (Table 6). Clinical remission at Week 14, based on endoscopic, stool frequency, and rectal bleeding subscores, was an exploratory endpoint. Using the MLE method on data from the FAS population, 9 participants (18.0% remission rate; Clopper-Pearson 95% CI 8.58-31.44) achieved clinical remission at Week 14. Another exploratory endpoint was change from baseline in partial Mayo score. A mean decrease from baseline was observed from Week 2 to Week 12, and this decrease was maintained until Week 26 (FIG. 3). Finally, 36/50 participants (72.0%) achieved clinical response at Week 14.

Of the 42 participants completing the study, 14 (33.3%) had RHI ≤5, and 20 (47.6%) had GI ≤3.2 at Week 14, demonstrating minimal histologic activity. Of the 50 participants enrolled, the proportion achieving histologic remission at Week 14 was 40.0% (GI ≤3.0 or RHI ≤6).

### PK and Immunogenicity

Following multiple IV 500 mg PF-06480605 doses Q2W for a maximum of 7 doses, maximum concentration ranged from 0.0993 to 1.24 mg/mL, followed by a biphasic decline with a half-life of 19 days. Total sTL1A concentration increased from baseline and peaked at a mean of 8267.5 pg/mL (range 744-20718 pg/mL) at Week 8, remaining higher than baseline up to Week 26. These results were indicative of target engagement, given that PF-06480605 binds to and stabilizes sTL1A, slowing its elimination.¹⁷ A significant reduction from baseline (51-78%) in fecal calprotectin was observed and sustained from Week 2 to Week 26. Similarly, a sustained reduction from baseline in hsCRP was observed from Week 2 to Week 26. A total of 41 participants (82.0%) were ADA-positive, and 5 (10.0%) were NAb-positive. A trend of lower target engagement was observed from Week 8 in ADA- and NAb-positive subjects compared with negative subjects.

### Example 2 Bioanalytical Methods

An Immunoprecipitation LC-MS/MS assay has been developed and validated to measure total soluble TL1A in human serum. Total soluble TL1A is isolated from human serum using immuno-enrichment technology. The samples are incubated and shaken with a biotinylated anti-TL1A capture reagent at 4° C. overnight. Dynabeads MyOne C1 streptavidin-coupled magnetic beads are then added to each sample and incubated at room temperature for 1 h to extract TL1A bound to biotinylated capture antibody. The beads are then washed three times, followed by elution of total TL1A from the beads under acidic conditions. After addition of the extended sequence stable isotope labeled peptide (internal standard) to each sample, trypsin digestion is performed at 37° C. overnight. All sample processing is performed in a 96-well format on an automated liquid handling robot (Microlab STAR, Hamilton, Bonaduz, Switzerland). A 120 μL of the sample extract is injected onto a three-dimensional Dionex Ultimate 3000 nano-LC system, comprising a conventional flow immunoaffinity capture with custom 2.1 mm ID anti-peptide antibody column, and elution to 300 μm ID C18 trap column and 75 μm ID nano LC analytical column. The signature peptide is eluted from the nano-LC column with a mobile phase gradient at a flow rate of 1 μL/min. A Thermo Vantage Triple Quadrupole mass spectrometer with Thermo Easy Spray ionization source is used for MS/MS analysis. One selected reaction monitoring transition for the signature peptide is used to quantify TL1A in the positive ion mode, and all data are normalized to the internal standard response. The validated analytical range is 40.0 to 6,000 pg/mL. The assay is precise and accurate with inter-batch imprecision <13.6% and inter-batch inaccuracy −6.0 to 0.5% at all concentrations investigated during assay validation.

An Immunoprecipitation LC-MS/MS assay has been developed and validated to measure free soluble TL1A in human colon tissue. Tissue samples are homogenized by Bead Beater and free TL1A is isolated from human colon tissue using immuno-enrichment technology. The samples are incubated and shaken with a biotinylated DcR3 capture reagent at 4° C. overnight. Dynabeads T1 streptavidin-coupled magnetic beads are then added to each sample and incubated at room temperature for 1 h to extract TLIA bound to biotinylated capture antibody. The beads are then washed three times, followed by elution of free TLIA from the beads under acidic conditions. After addition of the extended sequence stable isotope labeled peptide (internal standard) to each sample, trypsin digestion is performed at 37° C. overnight. All sample processing is performed in a 96-well format on an automated liquid handling robot (Microlab STAR, Hamilton, Bonaduz, Switzerland). A 120 μL of the sample extract is injected onto a three-dimensional Dionex Ultimate 3000 nano-LC system, comprising a conventional flow immunoaffinity capture with custom 2.1 mm ID anti-peptide antibody column, and elution to 300 μm ID C18 trap column and 75 μm ID nano LC analytical column. The signature peptide is eluted from the nano-LC column with a mobile phase gradient at a flow rate of 0.6 μL/min. A Thermo Vantage Triple Quadrupole mass spectrometer with Thermo Easy Spray ionization source is used for MS/MS analysis. One selected reaction monitoring transition for the signature peptide is used to quantify TL1A in the positive ion mode, and all data are normalized to the internal standard response. The validated analytical range is 10 to 400 pg/mL. The tissue assay is qualified and is precise and accurate with inter-batch imprecision <14.6% and inter-batch inaccuracy −0.8 to 6.0% at all concentrations investigated during assay validation.

Transcriptomic profiling of gut biopsies from UC patients were evaluated using RNA sequencing (RNASeq) technology All samples were extracted from blood and tissue and library were prepared by BGI Americas Corporation using GlobinClear+TrueSeq Stranded mRNA Sample Preparation Kit. Next generation sequencing was performed using the Illumina HiSeq4000 with a read length of 100PE resulting in 40 M reads.

Transcriptomics analysis was performed by estimating the fold change for the comparisons of inflamed and non-inflamed tissue at baseline and change from baseline, under the general framework for linear models using limma, voom packages. P-values from the paired t-test were adjusted for multiple hypotheses using the Benjamini-Hochberg procedure, which controls for FDR (Benjamini and Hochberg, 1990, Stat Med, July; 9(7):811-8). Differences in baseline gene expression between inflamed and non-inflamed biopsies were calculated. We also calculated change from baseline in responders and non-responders, using a linear mixed effect model using the factors of time and tissue response (defined as responder (R) and non-responder (NR)). This analysis is the used to correlate transcriptomic changes with clinical response. The primary efficacy endpoint was endoscopic index at Week 14, defined by a Mayo endoscopic subscore of 0 or 1 without friability. To ensure objective and consistent assessment of the primary endpoint, the Mayo endoscopic subscore was determined through blinded, centrally read colonoscopy images with built-in adjudications.

### Haplotype B and SNP Analyses

Haplotypes were phased using haplo.stats R package (Lake, et al 2003, Hum Hered.; 55(1):56-65) based on the genotyping data of five SNPs r53810936, r56478108, r56478109, rs7848647, rs7869487. Association analyses of haplotype B and SNP (allelic test) with binary outcomes was performed using Fisher's exact test. Association analysis of haplotype B and SNP with continuous outcomes was performed based on T-test.

### Protein Analysis

Protein profiling from blood were analyzed using Myriad/RBM Lab Menu and Myriad/RBM Simoa services. There are total of 63 proteins in the panel, after removing proteins with high rate of missing values (>50%). 52 out of 63 proteins are qualified to the downstream analysis. 10 out 63 proteins were measured by Myriad RBM Simoa™ Services. A linear mixed effect model was used to analyze the differences of log 2 transformed protein levels in blood between baseline and at weeks 2, 8 and 14 in responder and non-responder cohorts, respectively (The model takes participants as random effect, duration of treatment as fixed effect and also adjusts age, gender and smoke as covariates.). P-values were adjusted for multiple hypotheses using the Benjamini-Hochberg procedure, which controls for FDR, false discovery rate.

Baseline Inflamed Tissue Prediction Model and Validation with Permutation Experiment

Raw sequence counts were obtained from BGI RNA-seq and processed to Fragments Per Kilobase of transcript per Million mapped reads (FPKM) derived from all inflamed tissue samples at baseline. We use P-value ≤5E-4 to identify the candidate genes between responders and non-responders. The top 10 candidate genes were selected using non-parametric feature ranking algorithm in mlr R package (Bischl et al, Journal of Machine Learning Research, 17(170), 1-5) in order to limit overfitting. We then evaluated the prediction accuracies of the four models (Generalized Linear Model (GLMNET), Sparse Partial Least Square (SPLS), Support Vector Machine (SVM), and Random Forest (RF)) regarding to responders and non-responders. First, we generated receiver operating characteristic curves (ROCs) and estimated the areas under the curve (AUC) to summarize the predictive ability of endoscopic improvement given all selected genes and models, using on 5-fold cross-validations (CVs) with 5 replications. Next, we performed a random permutation test for all genes regardless of their R or NR status and ran the test over 200 times and evaluated the resultant AUCₚₑᵣₘs generated for each model. For each permutation, the procedure followed the feature selection, ranking and modeling steps as implemented in CV. The empirical p-value for the each of the model (Pvalue_{model}) is calculated as

$${Pvalue}_{model} = \frac{Σ I \{ {AUC}_{perm} \geq AUC \}}{200}$$

The smaller the Pvalue_{model}, the better it proves the significance of the model compared to random permutation experiment. R software were used for statistical modeling and analyses.
The permutation adjusted AUC is calculated as

Permutation Adjusted AUC_{model}=inverlogit(logit(AUC)−(Median(logit(AUCₚₑᵣₘ))−logit(0.5)))

This procedure adjusted the AUC from the observed data by shifting down by the appropriate transformation of the difference observed between the median of AUCₚₑᵣₘs and the expected null AUC of 0.5. we also calculated the adjusted AUC 95% CI from the 95% CI of the predictive model from observed data for all the methods using the permutation test and the same shift calculated above. Terms: Logit(x)=Log(x/(1−x))), inverlogit is defined as inverse of the logit(x) function as exp(x)/(1+exp(x)). Median(x) is the function of take the median of the input of vector x.

### Metagenomics Sample Collection and Processing

Stool samples were stored with preservative butter for stability of microbial content, DNA extraction following Enterome standards and quality control (Thomas et al 2015, Future Microbiol.; 10(9):1485-504). Next, whole metagenome sequencing was performed using Illumina platforms to achieve at least 40 million reads per sample. The quality of raw data in fastq files were assessed through FastQC ( ). The raw sequence was used for taxonomy classification by GOTTCHA 1.0c with the v20150825 bacterial database (GOTTCHA_BACTERIA_c4937_k24_u30_xHUMAN3x.species.tar.gz). The extracted result was normalized against the library size and differential abundance analysis was processed using a linear mixed-effects model with Ime function in R package nlme v3.1-143. P-value was adjusted by Benjamini-Hochberg method for the False Discovery Rate (FDR).

The beta diversity is estimated using 162 identified bacteria species using GOTTCHA pipeline. The bary.part function from R package betapart [Baselga, A. in press. Separating the two components of abundance-based dissimilarity: balanced changes in abundance vs. abundance gradients. Methods in Ecology and Evolution. DOI: 10.1111/2041-210X.12029] was used to calculate the Bray-Curtis dissimilarity among all the samples.

The betadisper function from R package vegan [Anderson, M. J. (2006) Distance-based tests for homogeneity of multivariate dispersions. Biometrics 62, 245-253] is used to assess the beta diversity by test for homogeneity of variances of samples based on the dissimilarities. P-value is calculated from ANOVA model of pre- and post-therapy samples.

### Reduction in Intestinal Th17, Th1, and Fibrosis Pathways in Anti-TL1A PF-06480605 Responders

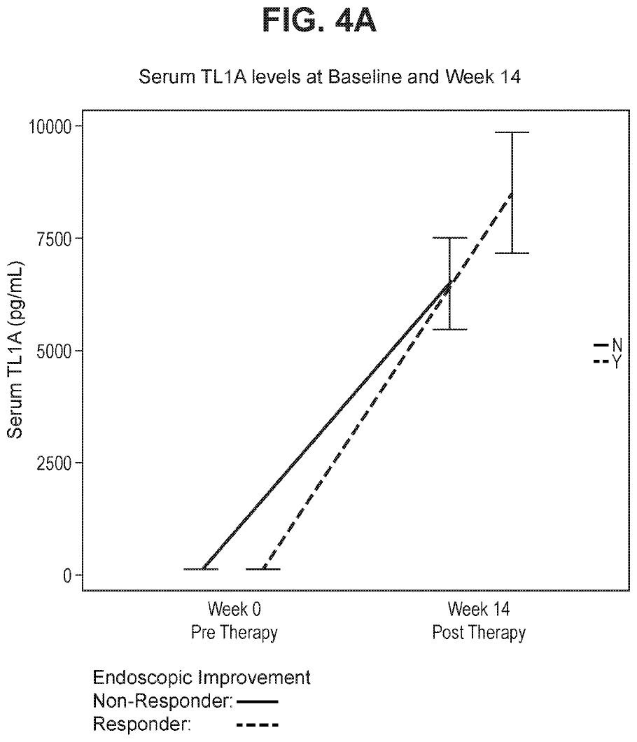

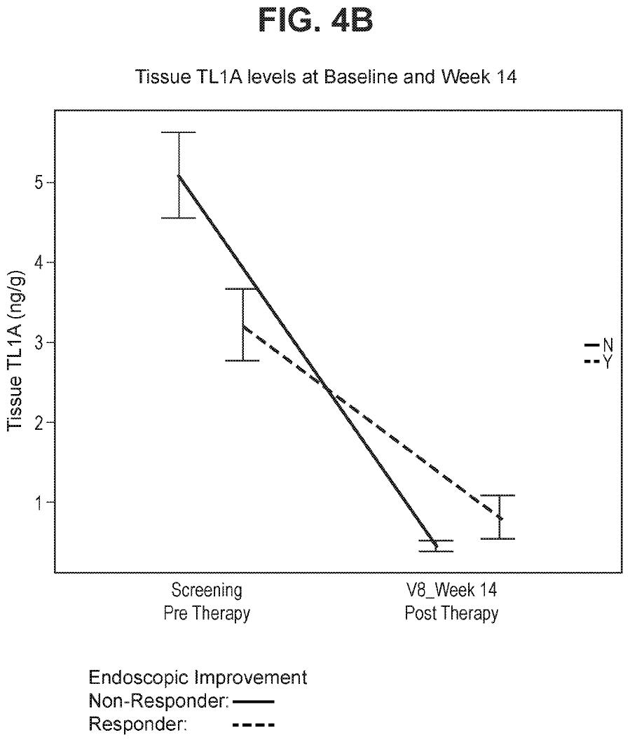

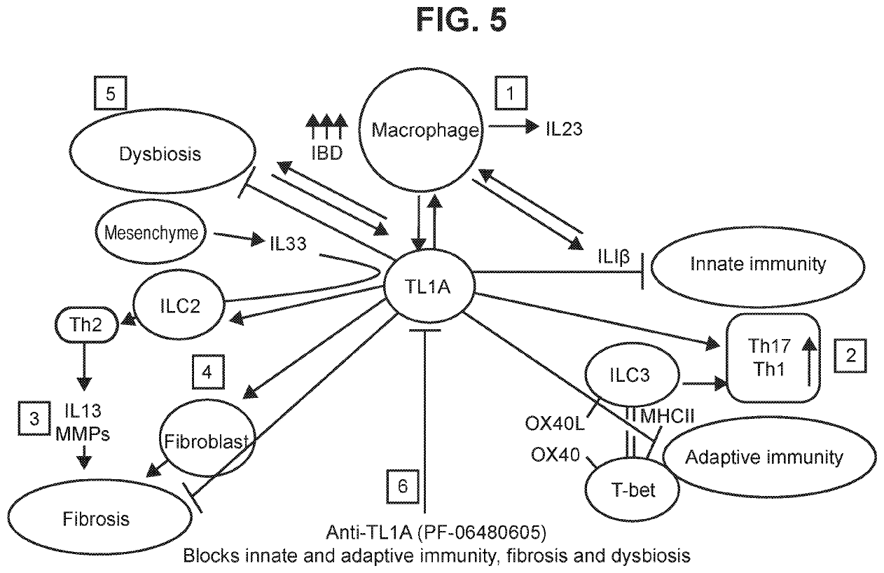

Given the clinical efficacy of anti-TL1A PF-06480605 to achieve endoscopic improvement in the treatment of refractory UC the underlying mechanism of this effect in intestinal tissue was evaluated. First, validated the specificity of anti-TL1A binding in vivo, by longitudinally measuring total TL1A (drug+TL1A) in the serum before and after therapy. The increase in total serum TL1A (FIG. 4A) reflects the stabilization of TL1A by PF-06480605 and the specificity of this binding. Next the impact of PF-06480605 in the intestine was assessed by measuring TL1A (FIG. 4B) levels in biopsy tissue before and after therapy. The significant reduction in tissue TL1A observed validates the efficacy of PF-06480605 in targeting tissue TL1A. Given the specificity and efficacy of PF-06480605 in targeting tissue TL1A, intestinal genes that are modulated after anti-TL1A therapy by performing RNA-sequencing of tissue biopsies from both inflamed and non-inflamed intestine were identified. differentially regulated gene sets were defined based on (a) disease activity (pre-treatment inflamed vs. non-inflamed intestine) (UC transcriptome) and (b) gene changes after anti-TL1A therapy (pre- vs post-treatment inflamed intestine (FC>2, FDR<0.05) (TL1A therapy transcriptome). Of the 565 differentially regulated genes identified in the treatment response transcriptome, 448 overlapping genes were identified in the UC transcriptome (UC-TL1A therapy transcriptome).

Within the TL1A-UC response transcriptome, we sought to identify mechanistic and cellular pathways associated with anti-TL1A response. In particular, the signature of response showed significant downregulation of IL-1B, IL-23A, IFNG, IL-12RB1, IL-21R, IRF4, and BATF, highlighting the potential impact of TL1A blockade on tissue Th17 and Th1 cells. Another subset of genes with downregulated expression in responders included: CD80/86, HLA-DRB5/DQB1/DRB1, HLA-DRA, CD40, ICOS, implicating a potential role for TL1A in recruitment and activation of antigen presenting cells in situ. In addition, several genes associated with remodeling of extracellular matrix and fibrosis were significantly downregulated with treatment response including: MMP3, MMP7, MMP10 and CHI3L (Table 7).

Using the set of 429 differentially expressed genes in treatment response, we performed Ingenuity Pathway Analysis (QIAGEN, CA) and identified the down regulation of signaling pathways (Z-score ≤−2) that target IL-17/IL-23 and Th1 as well PI3K, NF-kB, and ERK/MAPK. In addition, macrophage ROS, DC maturation, and Oncostatin M signaling pathways were significantly reduced in participants with or without endoscopic improvement. Finally, we gained insight into this tissue response at the cellular level, by performing deconvolution using CytoReason (Table 8) (J Gastroenterol. 2018 September; 53(9):1048-1064). Consistent with the gene and pathway analysis, CytoReason identified significant down regulation in the Th17 cell (FDR=2.55E-5), as well as, monocytes (FDR=0.0009), dendritic cells (FDR=0.0001), macrophages (FDR=0.0002), and memory B cell (FDR=0.0009) with endoscopic improvement.

Given the tissue specific impact of anti-TL1A, we sought to evaluate the potential for an immune biomarker of endoscopic improvement in the peripheral blood. Using the Myriad/RBM Simoa and custom platforms, we performed a differential analysis of week 2, 8, 14 from baseline and identified 20 out of 52 proteins with FDR <0.05 in participants with endoscopic improvement after anti-TL1A therapy. (Table 9). Concordant with the tissue transcriptomic results, proteomics analysis showed a significant decrease in IL-17A in both participants with endoscopic improvement (FDR=4.51E-11) and participants with no endoscopic improvement (FDR=9.23E-08), but with a more reduction in participants with endoscopic improvement after anti-TL1A therapy. In addition, peripheral blood analysis showed a robust reduction in type 2 associated cytokines IL-5 and IL-13 not reflected in the tissue transcriptional response. These results highlight the potential for peripheral blood markers to reflect a tissue transcriptional signature of reduced Th17 cell activation and a reduction in a systemic type 2 response that corresponds with endoscopic improvement.

### Fecal Metagenomics Defines Microbiome Changes Following Anti-TL1A PF-06480605 Therapy

Distinct changes exist in the active UC microbiome characterized by reduced diversity and an expansion in IBD-associated pathobionts. To characterize the bacterial species in the intestinal microbiome that are modulated in response to anti-TL1A therapy, we performed metagenomic sequencing of fecal DNA samples before and after treatment (Fig. S1). Principal Coordinate Analysis based on Bray-Curtis dissimilarities of samples from pre- and post-therapy show no significant change (Fig. S1) with a p-value of 0.53 using test of homogeneity of variances between pre- and post-anti-TL1A therapy. However, longitudinal abundance analysis (LAA) revealed significant change in bacteria species post treatment (Fig. S2). Using the GOTTCHA pipeline to assess differential abundance of taxa in all participants before and after treatment), we identified a significant decrease in the abundance in S. salivarius, (FDR=0.02), S. parasanguinis (FDR=0.02) and H. parainfluenzae (FDR=0.035) (Table 10). In contrast, an increase in SCFA-producing Ruminococcus and Bifidobacterium bifidum (with nominal P-values as 0.022 and 0.028, respectively) were increased following therapy (Table 10).

### TNFSF15 Haplotype and Tissue Transcriptome Predicts Treatment Response to Anti-TL1A PF-06480605 Therapy

Five single nucleotide polymorphisms (SNPs) (rs3810936, rs6478108, rs6478109, rs7848647, rs7869487) in TNFSF15 have been used to define three haplotypes (A, B, C). Haplotype A and C are associated with an increased risk for IBD, whereas haplotype B is significantly reduced in IBD patients (Thiebaut et al. Am J Gastroenterol. 2009; 104(2):384-91).

We found that haplotype B was enriched in non-responders (NR) with haplotype B frequency 25% vs. responders (R) with the haplotype frequency as 6.25% (P-value=0.04), but no association between haplotypes A and C were established. Both haplotype B and rs6478109 were not significantly associated with either TNFSF15 gene expressions or TL1A protein from inflamed tissues prior to treatment (Table 11).

Table 11: TNFSF15 Haplotype B was Enriched in Non-Responders (NR) Vs. Responders (R)
Haplotype analyses showed TNFSF15 haplotype B was enriched in non-responders (NR) vs. responders (R) with a nominal P=0.045, and haplotype B frequency of 18.18% in all participants.

| Haplotype | Count (Frequency) | Type | P-value |
|---|---|---|---|
| GACGA | 55 (62.5%) | A | 1 |
| AGTAG | 16 (18.18%) | B | 0.045 |
| AGTAA | 5 (5.68%) | C | 0.34 |

Given the tissue transcriptional signature of response (FIG. 4), we sought to determine if baseline tissue transcriptional signatures could predict endoscopic improvement after anti-TL1A therapy. We queried tissue transcriptional data from 49 samples (32 NR and 17 R) and selected the top 10 candidate genes by using non-parametric feature ranking algorithm in mlr R package (Table 12). We applied these 10 genes and created a prediction model of endoscopic response by using the four statistical methods (Random Forest (RF), Sparse Partial Linear Model (SPLS), Generalized Linear Model (GLMNET) and Support Vector Machine (SVM)) and permutation test. Using 200 random permutations of Responder/Non-responder status to gauge inflation from choosing the top 10 genes out of 15,000+, we can adjust the AUCs for overfitting while evaluating statistical significance as a permutation test. Under these adjustments, the predictive model with Random Forest algorithm resulted in the highest permutation-adjusted AUC of 0.71 with 95% CI (0.64-0.84), with a permutation-based p-value of p=0.02 (Table 12),

TABLE 5
UMVUE and MLE analyses of EI at Week 14 in participants treated
with PF-06480605 500 mg IV Q2W (PP, observed cases)

| | Endoscopic | | | Endoscopic | | |
|---|---|---|---|---|---|---|
| | subscore at | | | improvement rate | P-value^{a} | |
| Analysis | baseline | N | n | (%) | (one-sided Pr > Z) | 95% CI |
| UMVUE | 2/3 | 45 | 17 | 38.2 | <.001 | 23.82-53.68 |
| MLE | 2/3 | 45 | 17 | 37.8 | <.0001 | 23.77-53.46^{b} |
| | 2 | 5 | 3 | 60.0 | <.0001 | 14.66-94.73^{b} |
| | 3 | 40 | 14 | 35.0 | <.0001^{a} | 20.63, 51.68^{b} |

^{a}P-values were computed for the testing of the null hypothesis of 6% for EI rate at Week 14.
^{b}Clopper-Pearson method was used for 95% CI in the MLE analyses.
CI, confidence interval; EI, endoscopic improvement; IV, intravenous; MLE, maximum likelihood estimator; N, number of participants overall (PP population); n, number of participants with EI; PP, per protocol; Q2W, every 2 weeks; UMVUE, uniformly minimum-variance unbiased estimator.

TABLE 6
The proportion of participants treated with PF-06480605 500 mg
IV Q2W with remission and endoscopic remission at Week 14
(MLE analysis; FAS)

| | | | Remission | P-value^{a} | 95% Clopper- |
|---|---|---|---|---|---|
| Endpoint | N | n | rate (%) | (one-sided Pr > Z) | Pearson Cl |
| Remission | 50 | 12 | 24.0 | <.0001 | 13.06-38.17 |
| Endoscopic | 50 | 5 | 10.0 | =.1168 | 3.33-21.81 |
| remission | | | | | |

^{a}P-values were computed for the testing of the null hypothesis of 6% for EI rate at Week 14.
CI, confidence interval; EI, endoscopic improvement; FAS, full analysis set; IV, intravenous; MLE, maximum likelihood estimator; N, number of participants overall (PP population); n, number of participants with remission/endoscopic remission; PP, per-protocol; Q2W, every 2 weeks.

TABLE 7
Mechanistic and Cellular Pathways Impacting Inflammation and Fibrosis Genes are Associated with anti-TL1A Therapy
in Tissue: Significant genes in mechanistic and cellular pathways that were modulated in endoscopic improvement
inf. biopsy post Anti-TL1A therapy were identified by change from baseline with FDR <0.05 and |FC| >3

| | | | | Change from | Change from | Change from | Change from |
|---|---|---|---|---|---|---|---|
| | | | | Baseline | Baseline | Baseline | Baseline |
| | | | Baseline_infamed | (CFB) | (CFB) non- | (CFB) | (CFB) non- |
| | | | tissue/non- | inflamed | inflamed | inflamed | inflamed |
| | | | inflamed tissue | tissues in | tissues in | tissues in non- | tissues in non- |
| Pathway | gene | gene_name | FoldChange | Responders | Responders | responders | responders |
| Fibrosis | ENSG0005 | CHI3L1 | 1.46E+01 | −2.85E+01 | −6.10E+00 | −3.57E+00 | −1.71E+00 |
| Fibrosis/ | ENSG0003 | MMP7 | 1.42E+01 | −2.81E+01 | −3.99E+00 | −3.51E+00 | −1.35E+00 |
| inflammation | | | | | | | |
| Fibrosis | ENSG0004 | TCN1 | 1.79E+01 | −2.73E+01 | −5.25E+00 | −2.53E+00 | −2.08E+00 |
| Fibrosis/gut/ | ENSG0001 | MMP10 | 8.65E+00 | −1.60E+01 | −3.15E+00 | −3.59E+00 | −2.10E+00 |
| inflammation | | | | | | | |
| Fibrosis | ENSG0006 | ABCA12 | 1.64E+01 | −1.25E+01 | −3.18E+00 | −3.08E+00 | −1.03E+00 |
| IL23 Signaling/TH17 | ENSG00000125538.11 | IL1B | 1.04E+01 | −1.02E+01 | −2.51E+00 | −5.67E+00 | −1.24E+00 |
| IL23/TH1/TH2/TH17 | ENSG00000184557.4 | SOCS3 | 6.14E+00 | −9.25E+00 | −2.46E+00 | −3.83E+00 | −1.50E+00 |
| Fibrosis/leaky gut | ENSG0002 | PI3 | 1.15E+01 | −7.89E+00 | −1.76E+00 | −1.61E+00 | −1.09E+00 |
| IL23Signaling/TH17 | ENSG00000110944.8 | IL23A | 3.46E+00 | −6.58E+00 | −3.08E+00 | −2.34E+00 | −1.53E+00 |
| TH1/TH2 | ENSG00000090339.8 | ICAM1 | 3.67E+00 | −5.45E+00 | −2.51E+00 | −2.47E+00 | −1.30E+00 |
| TH2 | ENSG00000134460.15 | IL2RA | 2.09E+00 | −5.33E+00 | −2.51E+00 | −1.87E+00 | −1.45E+00 |
| IL23 Signaling/TH17 | ENSG00000049768.14 | FOXP3 | 3.35E+00 | −5.25E+00 | −2.27E+00 | −1.57E+00 | −1.13E+00 |
| TH17 | ENSG00000115009.11 | CCL20 | 2.33E+00 | −4.82E+00 | −2.32E+00 | −1.64E+00 | −1.04E+00 |
| TH1 | ENSG00000120217.13 | CD274 | 3.21E+00 | −4.78E+00 | −2.03E+00 | −2.33E+00 | −1.20E+00 |
| TH1/TH17 | ENSG00000255733.5 | IFNG | 3.27E+00 | −4.69E+00 | −2.07E+00 | −1.33E+00 | 1.00E+00 |
| TH17 | ENSG00000103522.15 | IL21R | 3.56E+00 | −4.09E+00 | −2.79E+00 | −1.43E+00 | 1.19E+00 |
| TH17 | ENSG00000137265.14 | IRF4 | 2.84E+00 | −3.91E+00 | −2.63E+00 | −1.45E+00 | −1.23E+00 |
| TH1/TH2 | ENSG00000121594.11 | CD80 | 2.67E+00 | −3.71E+00 | −2.14E+00 | −2.07E+00 | −1.05E+00 |
| TH1 | ENSG00000185338.4 | SOCS1 | 3.01E+00 | −3.70E+00 | −1.66E+00 | −1.84E+00 | −1.16E+00 |
| TH2 | ENSG00000163823.3 | CCR1 | 2.66E+00 | −3.64E+00 | −1.81E+00 | −2.35E+00 | −1.24E+00 |
| TH1/TH2 | ENSG00000198502.5 | HLA-DRB5 | 2.30E+00 | −3.62E+00 | −1.68E+00 | −1.37E+00 | −1.03E+00 |
| TH1/TH2 | ENSG00000179344.16 | HLA-DQB1 | 2.57E+00 | −3.43E+00 | −1.86E+00 | −1.34E+00 | 1.02E+00 |
| TH1/TH2 | ENSG00000163600.12 | ICOS | 2.42E+00 | −3.41E+00 | −2.48E+00 | −1.34E+00 | 1.08E+00 |
| TH2 | ENSG00000186827.10 | TNFRSF4 | 3.04E+00 | −3.39E+00 | −2.08E+00 | −1.51E+00 | −1.27E+00 |
| IL23 Signaling | ENSG00000232810.3 | TNF | 2.23E+00 | −3.39E+00 | −2.04E+00 | −1.84E+00 | −1.14E+00 |
| TH1/TH2 | ENSG00000204287.13 | HLA-DRA | 2.63E+00 | −3.31E+00 | −1.60E+00 | −1.42E+00 | 1.04E+00 |
| TH17 | ENSG00000156127.6 | BATF | 3.25E+00 | −3.18E+00 | −1.87E+00 | −1.93E+00 | −1.10E+00 |
| TH1/TH2/TH17 | ENSG00000105639.18 | JAK3 | 2.72E+00 | −3.14E+00 | −2.82E+00 | −1.48E+00 | 1.09E+00 |
| TH1/TH2 | ENSG00000074181.8 | NOTCH3 | 2.65E+00 | −3.03E+00 | −2.14E+00 | −1.26E+00 | −1.07E+00 |
| TH1/TH2 | ENSG00000160255.17 | ITGB2 | 2.57E+00 | −3.00E+00 | −2.05E+00 | −1.70E+00 | −1.07E+00 |

TABLE 8
CytoReason Deconvolution of Biopsy Transcriptomics Cell type estimation of CFB at week
14 in inf. and non-inf. biopsy using bulk RNA- seq in endoscopic improvement R and NR
using CytoReason deconvolution method. Cell types show with (*) have an FDR <0.05.

| | Differences_re- | Differences_re- | Differences_nonre- | Differences_nonre- |
|---|---|---|---|---|
| | sponders_in- | spoders_nonin- | sponders_in- | sponders_nonin- |
| TIS_CELL_TYPE | flamed_CFB | flamed_CFB | flamed_CFB | flamed_CFB |
| CD14-positive, CD16- | −1.21E−01 | −2.32E−02 | −9.27E−02 | −1.50E−02 |
| negative classical | | | | |
| monocyte | | | | |
| CD14-positive, CD16- | −1.39E−01 | −1.68E−02 | −8.27E−02 | −1.89E−02 |
| positive monocyte | | | | |
| CD16-negative, CD56- | 1.82E−02 | −1.26E−03 | −3.02E−03 | −7.52E−03 |
| bright natural killer cell | | | | |
| CD16-positive, CD56- | −6.55E−03 | 6.38E−03 | −1.51E−02 | 5.81E−03 |
| dim natural killer cell | | | | |
| CD4-positive, alpha-beta | −6.41E−02 | −4.82E−02 | −5.17E−02 | 1.66E−02 |
| T cell | | | | |
| CD8-positive, alpha-beta | −8.51E−03 | −1.28E−02 | 1.66E−02 | 2.42E−02 |
| T cell | | | | |
| central memory CD4- | −8.31E−02 | −6.96E−02 | −2.39E−02 | 1.91E−02 |
| positive, alpha-beta T | | | | |
| cell | | | | |
| central memory CD8- | 3.42E−02 | −1.23E−02 | 1.87E−02 | 2.15E−02 |
| positive, alpha-beta T | | | | |
| cell | | | | |
| dendritic cell | −1.56E−01 | −5.73E−02 | −6.51E−02 | −2.34E−02 |
| effector memory CD4- | −6.42E−04 | −2.46E−02 | −1.39E−02 | 9.80E−03 |
| positive, alpha-beta T | | | | |
| cell | | | | |
| effector memory CD8- | −2.21E−02 | −3.79E−02 | 6.56E−05 | 1.05E−02 |
| positive, alpha-beta T | | | | |
| cell | | | | |
| endothelial cell | −1.94E−01 | −7.93E−02 | −6.79E−02 | −1.89E−02 |
| eosinophil | −2.25E−02 | 3.71E−03 | −8.47E−03 | −4.99E−03 |
| epithelial cell | 4.83E−02 | −6.41E−03 | 7.85E−02 | −1.19E−04 |
| fat cell | 7.58E−02 | 1.76E−02 | 4.40E−02 | 8.28E−03 |
| fibroblast | −1.77E−01 | −8.75E−02 | −1.36E−01 | −4.50E−02 |
| gamma-delta Tcell | −7.35E−02 | −6.73E−02 | −5.47E−02 | −4.02E−03 |
| granulocyte | −2.07E−01 | −8.84E−02 | −2.36E−01 | −2.56E−02 |
| ILC1/3 | 1.15E−02 | −2.78E−02 | −1.70E−02 | 9.58E−03 |
| macrophage | −2.71E−01 | −6.60E−02 | −1.53E−01 | −6.57E−02 |
| mature B cell | −2.13E−01 | −1.90E−01 | 3.79E−02 | 2.25E−02 |
| mature natural killer cell | 5.88E−02 | −3.40E−03 | 1.92E−04 | 1.48E−02 |
| mature NKT cell | 2.85E−03 | −3.98E−02 | −1.21E−02 | 6.62E−03 |
| memory B cell | −1.18E−01 | −7.42E−02 | −5.07E−02 | −2.40E−02 |
| monocyte | −1.69E−01 | −3.70E−02 | −1.13E−01 | −3.29E−02 |
| myeloid dendritic cell | −1.89E−01 | −5.90E−02 | −5.52E−02 | −2.54E−02 |
| naive B cell | −1.52E−01 | −1.71E−01 | 7.00E−02 | 5.05E−02 |
| naive thymus-derived | 4.37E−02 | −2.78E−02 | 5.38E−02 | 9.52E−03 |
| CD4-positive, alpha-beta | | | | |
| T cell | | | | |
| naive thymus-derived | 1.43E−02 | 2.13E−02 | 4.25E−02 | 2.44E−02 |
| CD8-positive, alpha-beta | | | | |
| T cell | | | | |
| natural killer cell | 4.72E−03 | −8.15E−03 | −1.73E−02 | 1.66E−02 |
| neutrophil | −1.77E−01 | −7.89E−02 | −1.93E−01 | −1.19E−02 |
| plasmacytoid dendritic | −2.57E−02 | 6.62E−03 | −7.27E−03 | −2.51E−03 |
| cell | | | | |
| regulatory T cell | −7.60E−02 | −8.15E−02 | −5.03E−02 | 2.36E−02 |
| smooth muscle cell | −9.52E−02 | −7.95E−02 | −6.19E−02 | −2.18E−02 |
| T-helper 1 cell | −5.04E−02 | −4.04E−02 | −2.92E−02 | −2.23E−02 |
| T-helper 17 cell | −1.92E−01 | −7.36E−02 | −1.21E−01 | −2.23E−02 |
| T-helper 2 cell | −1.20E−02 | −4.61E−02 | 6.64E−03 | −4.76E−02 |

| | FDR_re- | FDR_re- | FDR_nonre- | FDR_nonre- |
|---|---|---|---|---|
| | sponders_in- | sponders_nonin- | sponders_in- | sponders_nonin- |
| TIS_CELL_TYPE | flamed_CFB | flamed_CFB | flamed_CFB | flamed_CFB |
| CD14-positive, CD16- | 1.46E−04 | 6.09E−01 | 6.90E−03 | 1.00E+00 |
| negative classical | | | | |
| monocyte | | | | |
| CD14-positive, CD16- | 8.94E−04 | 7.13E−01 | 7.00E−02 | 1.00E+00 |
| positive monocyte | | | | |
| CD16-negative, CD56- | 3.84E−02 | 8.70E−01 | 7.53E−01 | 1.00E+00 |
| bright natural killer cell | | | | |
| CD16-positive, CD56- | 7.04E−01 | 4.97E−01 | 8.90E−02 | 1.00E+00 |
| dim natural killer cell | | | | |
| CD4-positive, alpha-beta | 1.18E−03 | 1.79E−01 | 1.19E−01 | 1.00E+00 |
| T cell | | | | |
| CD8-positive, alpha-beta | 9.41E−01 | 7.13E−01 | 7.41E−01 | 1.00E+00 |
| T cell | | | | |
| central memory CD4- | 1.16E−02 | 2.56E−01 | 6.44E−01 | 1.00E+00 |
| positive, alpha-beta T | | | | |
| cell | | | | |
| central memory CD8- | 5.87E−02 | 4.97E−01 | 2.16E−01 | 1.00E+00 |
| positive, alpha-beta T | | | | |
| cell | | | | |
| dendritic cell | 1.44E−04 | 3.64E−01 | 4.68E−02 | 1.00E+00 |
| effector memory CD4- | 8.76E−01 | 3.83E−01 | 5.28E−01 | 1.00E+00 |
| positive, alpha-beta T | | | | |
| cell | | | | |
| effector memory CD8- | 9.89E−02 | 1.79E−01 | 1.00E+00 | 1.00E+00 |
| positive, alpha-beta T | | | | |
| cell | | | | |
| endothelial cell | 2.51E−03 | 4.97E−01 | 4.92E−01 | 1.00E+00 |
| eosinophil | 6.08E−01 | 8.91E−01 | 8.47E−01 | 1.00E+00 |
| epithelial cell | 6.20E−02 | 8.91E−01 | 3.50E−01 | 1.00E+00 |
| fat cell | 2.75E−03 | 7.22E−01 | 7.68E−02 | 1.00E+00 |
| fibroblast | 6.29E−03 | 3.64E−01 | 5.79E−02 | 1.00E+00 |
| gamma-delta Tcell | 1.35E−04 | 9.56E−02 | 8.26E−02 | 1.00E+00 |
| granulocyte | 8.94E−04 | 3.83E−01 | 5.98E−04 | 1.00E+00 |
| ILC1/3 | 5.17E−01 | 6.00E−01 | 3.97E−01 | 1.00E+00 |
| macrophage | 2.80E−04 | 3.90E−01 | 2.22E−02 | 1.00E+00 |
| mature B cell | 5.87E−02 | 3.83E−01 | 7.66E−01 | 1.00E+00 |
| mature natural killer cell | 5.83E−02 | 8.91E−01 | 8.36E−01 | 1.00E+00 |
| mature NKT cell | 7.80E−01 | 3.64E−01 | 6.68E−01 | 1.00E+00 |
| memory B cell | 8.94E−04 | 1.79E−01 | 2.02E−01 | 1.00E+00 |
| monocyte | 8.94E−04 | 4.97E−01 | 7.00E−02 | 1.00E+00 |
| myeloid dendritic cell | 1.66E−04 | 3.83E−01 | 7.68E−02 | 1.00E+00 |
| naive B cell | 1.18E−01 | 3.83E−01 | 6.08E−01 | 1.00E+00 |
| naive thymus-derived | 5.54E−02 | 3.83E−01 | 1.19E−01 | 1.00E+00 |
| CD4-positive, alpha-beta | | | | |
| T cell | | | | |
| naive thymus-derived | 4.32E−01 | 4.25E−01 | 4.68E−02 | 1.00E+00 |
| CD8-positive, alpha-beta | | | | |
| T cell | | | | |
| natural killer cell | 7.13E−01 | 7.13E−01 | 5.28E−01 | 1.00E+00 |
| neutrophil | 1.44E−04 | 2.98E−01 | 2.77E−04 | 1.00E+00 |
| plasmacytoid dendritic | 6.55E−02 | 8.70E−01 | 4.69E−01 | 1.00E+00 |
| cell | | | | |
| regulatory T cell | 1.54E−04 | 1.79E−01 | 1.33E−01 | 1.00E+00 |
| smooth muscle cell | 1.13E−01 | 3.64E−01 | 2.37E−01 | 1.00E+00 |
| T-helper 1 cell | 2.91E−02 | 4.97E−01 | 3.48E−01 | 1.00E+00 |
| T-helper 17 cell | 2.55E−05 | 1.79E−01 | 2.77E−04 | 1.00E+00 |
| T-helper 2 cell | 4.32E−01 | 7.13E−01 | 8.36E−01 | 1.00E+00 |

FIG. 9: Proteomics Identifies Inflammatory Blood Biomarkers of Endoscopic Improvement A total
52 out of 63 proteins were evaluated and significant proteins were selected based on CFB at
week 14 in endoscopic improvement R with FDR <0.05. All proteins with * are with FDR <0.05.

| | | LOG2_FC_re- | LOG2_FC_re- | LOG2_FC_re- | LOG2_FC_nonre- |
|---|---|---|---|---|---|
| Protein | Gene.Symbol | sponders_CFB_W 2 | sponders_CFB_W 8 | sponders_CFB_W 14 | sponders_CFB_W 2 |
| Interleukin-17A | IL17A | −1.43E+00 | −2.59E+00 | −2.42E+00 | −1.11E+00 |
| Simoa (IL-17A | | | | | |
| Simoa) | | | | | |
| Interleukin-5 | IL5 | −2.06E+00 | −2.13E+00 | −2.34E+00 | −7.51E−01 |
| Simoa (IL-5 | | | | | |
| Simoa) | | | | | |
| Interleukin-18 | IL18 | −2.36E−01 | −3.69E−01 | −4.29E−01 | 1.60E−02 |
| (IL-18) | | | | | |
| Matrix | MMP3 | −3.58E−01 | −4.95E−01 | −7.19E−01 | −1.44E−01 |
| Metalloprotein | | | | | |
| ase-3 (MMP-3) | | | | | |
| Interleukin-13 | IL13 | −1.24E+00 | −1.40E+00 | −1.39E+00 | −5.53E−01 |
| Simoa (IL-13 | | | | | |
| Simoa) | | | | | |
| C-Reactive | CRP | −1.88E+00 | −2.08E+00 | −1.60E+00 | −1.09E+00 |
| Protein (CRP) | | | | | |
| Thyroxine- | SERPINA7 | −1.99E−01 | −3.55E−01 | −2.77E−01 | −6.52E−02 |
| Binding | | | | | |
| Globulin (TBG) | | | | | |
| Alpha-2- | A2M | 4.80E−02 | 1.78E−01 | 3.08E−01 | 3.66E−02 |
| Macroglobulin | | | | | |
| (A2Macro) | | | | | |
| Vitronectin | VTN | −3.10E−01 | −2.73E−01 | −5.22E−01 | −2.20E−01 |
| Factor VII | F7 | −8.69E−02 | −1.54E−01 | −2.15E−01 | −9.21E−02 |
| Interleukin-6 | IL6 | −6.00E−01 | −1.20E+00 | −9.42E−01 | −3.28E−01 |
| Simoa (IL-6 | | | | | |
| Simoa) | | | | | |
| Serum Amyloid | APCS | −3.37E−01 | −3.70E−01 | −3.36E−01 | −1.56E−01 |
| P-Component | | | | | |
| (SAP) | | | | | |
| Tumor necrosis | TNFRSFIB | −1.73E−01 | −2.81E−01 | −3.30E−01 | −1.02E−01 |
| factor receptor | | | | | |
| 2 (TNFR2) | | | | | |
| Growth- | CXCL1 | −2.23E−01 | −4.72E−01 | −4.21E−01 | −3.57E−01 |
| Regulated | | | | | |
| alpha protein | | | | | |
| (GRO-alpha) | | | | | |
| Matrix | MMP9 | −4.26E−01 | −3.45E−01 | −4.68E−01 | −7.49E−02 |
| Metalloprotein | | | | | |
| ase-9, total | | | | | |
| (MMP-9, total) | | | | | |
| Vascular | VEGFC | −3.83E−01 | −4.25E−01 | −4.41E−01 | −2.52E−01 |
| Endothelial | | | | | |
| Growth Factor | | | | | |
| (VEGF) | | | | | |
| Interleukin-8 | CXCL8 | −5.59E−01 | −5.43E−01 | −8.28E−01 | −4.60E−01 |
| (IL-8) | | | | | |
| Tumor Necrosis | TNF | −1.33E−01 | −3.68E−01 | −2.81E−01 | −2.13E−01 |
| Factor alpha | | | | | |
| Simoa (TNFa | | | | | |
| Simoa) | | | | | |
| von Willebrand | VWF | −1.91E−01 | −4.19E−01 | −2.87E−01 | 2.17E−02 |
| Factor (vWF) | | | | | |
| Interleukin-9 | IL9 | −3.58E−01 | −3.83E−01 | −3.57E−01 | −3.31E−01 |
| Simoa (IL-9 | | | | | |
| Simoa) | | | | | |
| Pulmonary and | CCL18 | −1.81E−01 | −3.35E−01 | −1.90E−01 | −2.13E−01 |
| Activation- | | | | | |
| Regulated | | | | | |
| Chemokine | | | | | |
| (PARC) | | | | | |

| | LOG2_FC_nonre- | LOG2_FC_nonre- | FDR_re- | FDR_re- |
|---|---|---|---|---|
| Protein | sponders_CFB_W 8 | sponders_CFB_W 14 | sponders_CFB_W 2 | sponders_CFB_W 8 |
| Interleukin-17A | −1.58E+00 | −1.58E+00 | 1.74E−05 | 4.21E−12 |
| Simoa (IL-17A | | | | |
| Simoa) | | | | |
| Interleukin-5 | −1.15E+00 | −8.52E−01 | 1.58E−05 | 2.15E−06 |
| Simoa (IL-5 | | | | |
| Simoa) | | | | |
| Interleukin-18 | −2.03E−01 | −2.05E−01 | 1.21E−02 | 4.37E−05 |
| (IL-18) | | | | |
| Matrix | −3.14E−01 | −5.21E−01 | 4.06E−02 | 1.53E−03 |
| Metalloprotein | | | | |
| ase-3 (MMP-3) | | | | |
| Interleukin-13 | −6.95E−01 | −4.72E−01 | 7.29E−04 | 7.06E−05 |
| Simoa (IL-13 | | | | |
| Simoa) | | | | |
| C-Reactive | −1.59E+00 | −1.48E+00 | 1.58E−05 | 1.72E−06 |
| Protein (CRP) | | | | |
| Thyroxine- | −1.35E−01 | −2.10E−01 | 1.00E−02 | 2.15E−06 |
| Binding | | | | |
| Globulin (TBG) | | | | |
| Alpha-2- | 5.44E−02 | 1.45E−01 | 7.20E−01 | 3.01E−02 |
| Macroglobulin | | | | |
| (A2Macro) | | | | |
| Vitronectin | −2.81E−01 | −3.95E−01 | 5.60E−02 | 6.19E−02 |
| Factor VII | −4.91E−02 | −1.72E−01 | 2.60E−01 | 1.80E−02 |
| Interleukin-6 | −5.84E−01 | −1.82E−01 | 7.16E−02 | 1.15E−04 |
| Simoa (IL-6 | | | | |
| Simoa) | | | | |
| Serum Amyloid | −2.61E−01 | −1.89E−01 | 5.05E−03 | 9.66E−04 |
| P-Component | | | | |
| (SAP) | | | | |
| Tumor necrosis | −2.43E−01 | −1.68E−01 | 1.86E−01 | 1.49E−02 |
| factor receptor | | | | |
| 2 (TNFR2) | | | | |
| Growth- | −3.49E−01 | −2.89E−01 | 2.02E−01 | 1.73E−03 |
| Regulated | | | | |
| alpha protein | | | | |
| (GRO-alpha) | | | | |
| Matrix | −5.63E−02 | −1.47E−01 | 2.25E−02 | 4.20E−02 |
| Metalloprotein | | | | |
| ase-9, total | | | | |
| (MMP-9, total) | | | | |
| Vascular | −1.48E−01 | −2.42E−01 | 4.21E−02 | 1.36E−02 |
| Endothelial | | | | |
| Growth Factor | | | | |
| (VEGF) | | | | |
| Interleukin-8 | −7.38E−01 | −2.13E−01 | 1.60E−01 | 9.80E−02 |
| (IL-8) | | | | |
| Tumor Necrosis | −3.25E−01 | −9.40E−02 | 3.39E−01 | 1.42E−03 |
| Factor alpha | | | | |
| Simoa (TNFa | | | | |
| Simoa) | | | | |
| von Willebrand | −3.62E−01 | −3.81E−01 | 1.86E−01 | 8.78E−04 |
| Factor (vWF) | | | | |
| Interleukin-9 | −3.75E−01 | −1.96E−01 | 4.24E−02 | 1.57E−02 |
| Simoa (IL-9 | | | | |
| Simoa) | | | | |
| Pulmonary and | −2.92E−01 | −2.12E−01 | 5.17E−02 | 1.22E−04 |
| Activation- | | | | |
| Regulated | | | | |
| Chemokine | | | | |
| (PARC) | | | | |

| | FDR_re- | FDR_nonre- | FDR_nonre- | FDR_nonre- |
|---|---|---|---|---|
| Protein | sponders_CFB_W 14 | sponders_CFB_W 2 | sponders_CFB_W 8 | sponders_CFB_W 14 |
| Interleukin-17A | 4.51E−11 | 2.95E−04 | 5.15E−08 | 9.23E−08 |
| Simoa (IL-17A | | | | |
| Simoa) | | | | |
| Interleukin-5 | 6.86E−07 | 7.32E−02 | 2.53E−03 | 3.16E−02 |
| Simoa (IL-5 | | | | |
| Simoa) | | | | |
| Interleukin-18 | 3.82E−06 | 8.88E−01 | 4.20E−03 | 6.96E−03 |
| (IL-18) | | | | |
| Matrix | 1.23E−05 | 3.58E−01 | 1.85E−02 | 4.45E−04 |
| Metalloprotein | | | | |
| ase-3 (MMP-3) | | | | |
| Interleukin-13 | 9.19E−05 | 3.06E−02 | 1.66E−03 | 3.87E−02 |
| Simoa (IL-13 | | | | |
| Simoa) | | | | |
| C-Reactive | 1.07E−04 | 3.06E−02 | 4.28E−04 | 1.69E−03 |
| Protein (CRP) | | | | |
| Thyroxine- | 1.34E−04 | 3.56E−01 | 1.59E−02 | 5.99E−04 |
| Binding | | | | |
| Globulin (TBG) | | | | |
| Alpha-2- | 2.77E−04 | 6.98E−01 | 4.46E−01 | 3.43E−02 |
| Macroglobulin | | | | |
| (A2Macro) | | | | |
| Vitronectin | 5.36E−04 | 7.69E−02 | 1.20E−02 | 8.34E−04 |
| Factor VII | 1.17E−03 | 2.54E−01 | 4.96E−01 | 1.60E−02 |
| Interleukin-6 | 2.12E−03 | 3.56E−01 | 4.87E−02 | 5.75E−01 |
| Simoa (IL-6 | | | | |
| Simoa) | | | | |
| Serum Amyloid | 2.58E−03 | 4.51E−02 | 2.98E−04 | 1.23E−02 |
| P-Component | | | | |
| (SAP) | | | | |
| Tumor necrosis | 4.56E−03 | 3.56E−01 | 8.93E−03 | 7.08E−02 |
| factor receptor | | | | |
| 2 (TNFR2) | | | | |
| Growth- | 5.62E−03 | 3.36E−02 | 1.81E−02 | 6.57E−02 |
| Regulated | | | | |
| alpha protein | | | | |
| (GRO-alpha) | | | | |
| Matrix | 5.62E−03 | 5.85E−01 | 6.24E−01 | 1.74E−01 |
| Metalloprotein | | | | |
| ase-9, total | | | | |
| (MMP-9, total) | | | | |
| Vascular | 9.30E−03 | 1.06E−01 | 3.24E−01 | 8.94E−02 |
| Endothelial | | | | |
| Growth Factor | | | | |
| (VEGF) | | | | |
| Interleukin-8 | 1.47E−02 | 3.06E−02 | 1.02E−04 | 2.70E−01 |
| (IL-8) | | | | |
| Tumor Necrosis | 1.58E−02 | 3.06E−02 | 2.98E−04 | 2.99E−01 |
| Factor alpha | | | | |
| Simoa (TNFa | | | | |
| Simoa) | | | | |
| von Willebrand | 1.99E−02 | 9.28E−01 | 2.22E−02 | 2.80E−02 |
| Factor (vWF) | | | | |
| Interleukin-9 | 2.28E−02 | 4.51E−02 | 1.25E−02 | 2.24E−01 |
| Simoa (IL-9 | | | | |
| Simoa) | | | | |
| Pulmonary and | 2.64E−02 | 3.06E−02 | 1.03E−03 | 2.34E−02 |
| Activation- | | | | |
| Regulated | | | | |
| Chemokine | | | | |
| (PARC) | | | | |

TABLE 10
Reduction in intestinal Pathobiome Post Anti-TL1A therapy

| | linear.depth. | | |
|---|---|---|---|
| Bacteria | coverage.FC | p.value | FDR |
| Streptococcus salivarius | −7.12E−01 | 2.25E−04 | 2.03E−02 |
| Streptococcus parasanguinis | −8.60E−01 | 2.50E−04 | 2.03E−02 |
| Haemophilus parainfluenzae | −1.25E+00 | 6.52E−04 | 3.52E−02 |
| Gordonibacter pamelaeae | −6.59E−01 | 2.87E−03 | 1.16E−01 |
| Lactococcus lactis | −7.82E−01 | 5.22E−03 | 1.69E−01 |
| Eggerthella lenta | −8.06E−01 | 9.29E−03 | 2.42E−01 |
| Streptococcus anginosus | −1.41E+00 | 1.04E−02 | 2.42E−01 |
| Streptococcus sp. I-P16 | −8.90E−01 | 1.76E−02 | 2.97E−01 |
| Streptococcus mutans | −9.64E−01 | 1.82E−02 | 2.97E−01 |
| Lactobacillus salivarius | −2.20E+00 | 1.83E−02 | 2.97E−01 |
| Ruminococcus champanellensis | 1.11E+00 | 2.17E−02 | 3.19E−01 |
| Odoribacter splanchnicus | −8.43E−01 | 2.83E−02 | 3.55E−01 |
| Bifidobacterium bifidum | 9.25E−01 | 2.85E−02 | 3.55E−01 |
| Leuconostoc carnosum | −1.69E+00 | 3.51E−02 | 4.03E−01 |
| Haemophilus influenzae | −1.28E+00 | 3.73E−02 | 4.03E−01 |
| Aggregatibacter aphrophilus | −3.17E+00 | 4.18E−02 | 4.23E−01 |

Bacteria species identified that CFB (P-value <= 0.05), −log10(p-value) = 1.3 in fecal samples in all participants. The top three bacteria species (*) are based on the threshold using FDR <= 0.05.

1. A method for treating inflammatory bowel disease (IBD) in a patient, the method comprising administering to the patient an anti-TNF-like ligand 1A (TL1A) antibody in a induction dosing regimen sufficient to improve signs and symptoms of IBD by at least 12 weeks after the start of treatment with the anti-TL1A antibody, said induction dosing regimen comprising a plurality of individual induction doses, wherein the method further comprises administering to the patient a subsequent maintenance dosing regimen after completion of the induction dosing regimen, said maintenance dosing regimen comprising a plurality of individual maintenance doses separated from each other by at least 2 weeks.

2. The method as set forth in claim 1, wherein the individual maintenance doses are administered at least 1, 2, 3, 4, or 6 months apart.

3. The method as set forth in claim 1, wherein the individual maintenance doses are no more than about 75% of the individual induction doses.

4. The method as set forth in claim 1, wherein the individual induction dose is about 500 mg via intravenous injection.

5. The method as set forth in claim 1, wherein the individual induction doses are separated from each other by at least 2 weeks.

6. The method according to claim 1, wherein the IBD is ulcerative colitis (UC).

7. The method for treating inflammatory bowel disease (IBD) in a patient, the method comprising the steps of:
a) determining the expression level of one or more candidate genes in a sample from the patient,
b) identifying that the sample contains an abnormal expression level of the one of more candidate gene,
c) administering an induction dose of an anti-TNF-like ligand 1A (TL1A) antibody to the patient.

8. The method as set forth in claim 7, wherein the one or more candidate genes is selected from the group consisting of SOWAHB, COLCA2, TBX20, FRZB, HOXB5, NET1, FOXD2, DESI1, PARK2, PKDREJ, IL-1B, IL-23A, IFNG, IL-12RB 1, IL-21R, IRF4, BATF, CD 80/86, HLA-DRB5/DQB1/DRB1, HLA-DRA, CD40, ICOS, MMP3, MMP7, MMP10, and CHI3L.

9. The method as set forth in claim 7, wherein the one or more candidate genes are selected from the group consisting of SOWAHB, COLCA2, TBX20, FRZB, HOXB5, NET1, FOXD2, DESI1, PARK2, and PKDREJ.

10. The method as set forth in claim 7, wherein the expression level of the one or more candidate gene is compared against a baseline expression level which is
a) based on the expression level of the one or more candidate gene for a healthy individual who is not suffering from IBD or UC; or
b) based on an estimated expression level for individuals who are non-responsive to anti-TL1A antibody treatment.

11. The method as set forth in claim 7, wherein the abnormal expression level of the one or more candidate gene is at least 50% greater or lesser from the baseline level.

12. The method as set forth in claim 7, the method comprising the steps of:
(a) determining whether the patient is a haplotype A, B or C for TNFSF15 by obtaining a biological sample from the patient;
(b) performing a genotyping assay on the biological sample to determine if the patient is of haplotype A, B or C for TNFSF15; wherein the risk of the patient being non-responsive to the therapeutic dose of anti-TL1A antibody is lower in a patient of haplotype A or haplotype C than in a patient of haplotype B;
wherein if the patient is of haplotype B for TNFSF15, a maintenance dosage regimen of the anti-TL 1A antibody is administered to the patient wherein the maintenance dose provides an increased individual maintenance dose relative to the individual maintenance dose provided to patients of haplotype A or C; and
wherein if the patient is of haplotype B for TNFSF15 then administering a maintenance dosage regimen of the anti-TL1A antibody to the patient that provides a decreased time interval between the individual maintenance doses relative to the time intervals between individual maintenance doses provided to patients of haplotype A or C.

13. A method for treating inflammatory bowel disease (IBD) in a patient, the method comprising the steps of:
(i) determining the level of one or more candidate bacterial strains in a stool sample from the patient,
(ii) identifying that the stool sample contains an increased or decreased level of the one of more candidate bacterial strains,
(iii) administering a therapeutic dose of an anti-TNF-like ligand 1A (TL1A) antibody to a patient.

14. The method as set forth in claim 13, wherein the candidate bacterial strain level is increased, and the candidate bacterial strain is selected from the group consisting of Streptococcus salivarius, Streptococcus parasanguinis, and Haemophilus parainfluenzae.

15. The method as set forth in claim 14, wherein the candidate bacterial strain level is decreased, and the candidate bacterial strain is selected from the group consisting of Ruminococcus albus, Ruminococcus callidus, Ruminococcus bromii, Ruminococcus gnavus, and Bifidobacterium bifidum.

16. The method according to claim 1, wherein the anti-TL1A antibody comprises three CDRs from the variable heavy chain region having the sequence shown in SEQ ID NO: 1 and three CDRs from the variable light chain region having the sequence shown in SEQ ID NO: 2.

17. The method according to claim 1, wherein the anti-TL1A antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

18. The method according to claim 1, wherein the anti-TL1A antibody comprises a variable heavy chain region having the sequence shown in SEQ ID NO: 1 and a variable light chain region having the sequence shown in SEQ ID NO: 2.

19. The method according to claim 1, wherein the anti-TL1A antibody comprises sequence pairs selected from the group consisting of SEQ ID NO:4 and 11; SEQ ID NO:4 and 12; SEQ ID NO:4 and 13; SEQ ID NO:4 and 14; SEQ ID NO:4 and 15; SEQ ID NO:4 and 16; SEQ ID NO:4 and 17; SEQ ID NO:4 and 18; SEQ ID NO:4 and 19; SEQ ID NO:20 and 24; SEQ ID NO:21 and 25; SEQ ID NO:22 and 26; SEQ ID NO:23 and 27; SEQ ID NO:28 and 29; SEQ ID NO:30 and 31; and SEQ ID NO:30 and 31.

20. The method as claimed in claim 1, further comprising treatment with an IL-23 antagonist.

21. The method as set forth in claim 1, wherein the patient has moderate to severe ulcerative colitis.

22. Use of a compound for the preparation of a medicament for the treatment of IBD according to a method of claim 1.

23. The method according to claim 7, wherein the anti-TL1A antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

24. The method according to claim 7, wherein the anti-TL1A antibody comprises a variable heavy chain region having the sequence shown in SEQ ID NO: 1 and a variable light chain region having the sequence shown in SEQ ID NO: 2.

25. The method according to claim 13, wherein the anti-TL1A antibody comprises a HCDR1 having the sequence shown in SEQ ID NO:3, a HCDR2 having the sequence shown in SEQ ID NO:4, a HCDR3 having the sequence shown in SEQ ID NO:5, a LCDR1 having the sequence shown in SEQ ID NO:6, a LCDR2 having the sequence shown in SEQ ID NO:7, and a LCDR3 having the sequence shown in SEQ ID NO:8.

26. The method according to claim 13, wherein the anti-TL1A antibody comprises a variable heavy chain region having the sequence shown in SEQ ID NO: 1 and a variable light chain region having the sequence shown in SEQ ID NO: 2.
